(19) United States
(12) Patent Application Publication
(10) Pub. No.: US 2022/0265741 A1
(43) Pub. Date: Aug. 25, 2022

(54) Novel oncolytic parvoviruses with enhanced cargo capacity, stable shRNA expression cassette and novel immunogenic properties

(71) Applicant: Alliance of Cardiovascular Researchers, Houston, TX (US)

(72) Inventors: Antonio MARCHINI, Luxembourg (LU); Tiina MARTTILA, Luxembourg (LU); Amit KULKARNI, Luxembourg (LU); Eckhard U. ALT, Houston, TX (US)

(21) Appl. No.: 17/670,789

(22) Filed: Feb. 14, 2022

Related U.S. Application Data
(60) Provisional application No. 63/149,217, filed on Feb. 12, 2021.

(51) Int. Cl. A61K 35/768; C12N 7/00; C12N 15/86; C12N 15/113; A61K 31/7105; A61P 35/00

(52) U.S. Cl. CPC A61K 35/768; C12N 7/00; C12N 15/86; C12N 15/1135; A61K 31/7105; C12N 2750/14332; C12N 2310/531; C12N 2310/111; C12N 2750/14322; C12N 2750/14342; A61P 35/00

(57) ABSTRACT

Novel engineered oncolytic protoparvoviruses are described to be used in cancer therapy. The engineered protoparvoviruses contain at least one deletion in the untranslated region and a silencer sequence that remains stably integrated into the viral genome during extensive virus propagation. The novel viruses can be used for the silencing of relevant cancer-related genes, providing to the virus a new anticancer mechanism of action.

### PRIOR RELATED APPLICATIONS

This application claims priority to U.S. provisional application Ser. No. 63/149,217, filed Feb. 12, 2021, which is incorporated herein in its entirety for all purposes.

### FEDERALLY SPONSORED RESEARCH STATEMENT

Not applicable.

### FIELD OF THE DISCLOSURE

The disclosure generally relates to novel parvoviruses and more particularly to novel engineered oncolytic protoparvoviruses harboring a shRNA expression cassette in their genomes.

### BACKGROUND OF THE DISCLOSURE

Oncolytic viruses (OVs) are viruses that have the capacity to infect and selectively destroy cancer cells without harming normal cells. The primary mechanism of action of oncolytic viruses was traditionally thought to be their inherent ability to replicate within, and ultimately lyse, cancer cells while sparing normal cells. The promising therapeutic efficacy of such viruses was demonstrated by their ability to lyse human cancer cells in culture and cause regression of human tumor xenografts (Russell, Peng, & Bell, 2012). With these findings, multiple oncolytic viruses have entered the clinical arena over the past 60 years, though the clinical results have not been as impressive, mostly due to the rapid development of neutralizing antibodies by the host.

Besides, OVs can induce and stimulate strong anti-tumor immune response and destroy tumor vasculature. As a result, not less than forty OVs are currently evaluated in clinical trials against a variety of different cancers as a monotherapy or in combination with other anticancer agents. For example, rat protoparvovirus H-1PV has been under clinical development. Phase I/IIa clinical trial in patients with glioblastoma showed that H-1PV virus treatment is safe, well tolerated and associated with first evidence of efficacy.

However, the wild type virus in the regimes used was unable to eradicate the tumor, indicating that further development is necessary to improve clinical outcome.

Additionally, there is the need for novel oncolytic viruses with improved replication efficiency as well as ease of genetic editing for further manipulation.

### SUMMARY OF THE DISCLOSURE

Therefore, in one aspect of this disclosure, an engineered parvovirus is described herein, where the parvovirus has a left palindromic terminal sequence, a coding sequence, a non-coding region, and a right palindromic terminal sequence. The coding sequence comprises a first gene unit encoding non-structural proteins (NS1 and NS2) under the control of the P4 viral early promoter and a second gene unit encoding viral capsid proteins (VP1 and VP2) and the non-structural SAT protein under the control of the P38 viral late promoter. The novel viruses also feature deletions into their genome, namely between the first sequence and the second sequence and deletions within the non-coding region. These novel viruses have increased cargo capacity, therefore capable of stably retaining in their genome a heterologous sequence, such as a silencing cassette, while keeping all other properties unchanged. For example, a stable insertion and replication of the silencing cassette is achieved in these novel viruses without compromising the ability of the virus to replicate and exert oncolytic activities.

In one embodiment, the engineered parvovirus is a protoparvovirus that includes H-1PV, LuIII, MVM and MPV.

In one embodiment, the parvovirus here termed AAH-1PV-v1, comprising a first deletion at nucleotide 2022 to 2135 of the wildtype H-1PV genome, and a second deletion at nucleotide 4659 to 4693 of the wild type H-1PV (Ref. Seq of the wild type H-1PV is Gene bank: X01457.1).

In one embodiment, the silencer cassette is inserted into the non-coding region. In certain embodiments, the silencer cassette is inserted at position 4659 of the parvoviral genome.

In one embodiment, the silencer cassette is inserted in the untranslated region of the LuIII genome. In certain embodiments, the silencer cassette is inserted at position 4575 of the LuIII genome.

In one embodiment, the silencer expressing sequence is a shRNA expressing cassette. In one embodiment, the shRNA expressing cassette comprises a sense sequence of a target gene, a loop, and an antisense sequence of the target gene.

In one embodiment the silencer expression cassette includes a promoter or promoter region regulated by a RNA polymerase II or a RNA polymerase III. In one embodiment the RNA-polymerase III promoter is the H1 promoter.

In one embodiment the shRNA sequence silences the expression of a gene related to cancer, e.g. oncogenes, anti-apoptotic genes, a gene critical for tumor cell growth, metastasis, acquisition of drug resistance, angiogenesis or aberrant expression of an immunomodulatory gene or a gene encoding an immunomodulatory checkpoint, cytokine, growth factor, enzyme or transcription factor.

In one embodiment, the target is a portion of TRAF3IP2. TRAF3IP2 encodes a protein involved in regulating responses to cytokines by members of the Rel/NF-κB transcription factor family. These factors play a central role in innate immunity in response to pathogens, inflammatory signals and stress. This gene product interacts with TRAF proteins (tumor necrosis factor receptor-associated factors) and either I-κB kinase or MAP kinase to activate either NF-κB or Jun kinase. Several alternative transcripts encoding different isoforms have been identified. Another transcript, which does not encode a protein and is transcribed in the opposite orientation, has been identified. Overexpression of this transcript has been shown to reduce expression of at least one of the protein encoding transcripts, suggesting it has a regulatory role in the expression of this gene.

In another aspect of this disclosure, a composition for treating a cancer is described. The composition comprises an effective amount of the engineered virus described herein, and a pharmaceutically acceptable carrier.

In one embodiment, the composition is formulated for intravascular injection, parenteral administration, intranasal administration or for intratumoral administration.

In one embodiment, the cancer is glioblastoma.

In one embodiment the novel viruses are used for the elimination of cancer stem-like cells.

In one embodiment, the silencer expression sequence in the engineered parvovirus is a shRNA expression cassette comprising a sense sequence of a target, a loop, and an antisense sequence of the target, and the target may be a portion of TRAF3IP2.

In another aspect of this disclosure, a composition for treating cancer is described. The composition comprises effective amount of the engineered LuIII parvovirus described herein, and a pharmaceutically acceptable carrier.

In another aspect of this disclosure, a method of treating a cancer is described. The method comprises administering to a subject an effective amount of the engineered parvovirus described herein, and a pharmaceutically acceptable carrier.

In one embodiment, the method further comprises, that the engineered parvoviruses described herein are used in combination with other anticancer agents (for instance but not exclusively with chemotherapy, radiotherapy, small molecule inhibitors, immunotherapy etc).

In one embodiment, after administering the engineered parvoviruses described herein, administering to the subject an effective amount of another oncolytic virus (the list includes herpes simplex virus, adenovirus, vaccinia virus, measles virus, coxsackie virus, poliovirus, reovirus, Newcastle disease virus, vesicular stomatitis virus, Seneca Valley virus) or a virus used in gene therapy (e.g. lentivirus or Adenovirus or AAV).

In one embodiment, after administering the engineered parvoviruses described herein, administering to the subject an effective amount of another oncolytic protoparvovirus.

In one embodiment, the method further comprises, after administering the engineered H-1PV parvovirus described herein, administering to the subject an effective amount of engineered LuIII parvovirus described herein.

In one embodiment, the method further comprises, after administering the engineered LuIII parvovirus described herein, administering to the subject an effective amount of engineered H-1PV parvovirus described herein. In one embodiment, the engineered LuIII and H-1PV parvoviruses can be administered co-sequentially or simultaneously.

In one embodiment, the two engineered parvoviruses are used in combination for the silencing of the same target gene, or they can silence different target genes. The two engineered parvoviruses can have different functions in treating cancer, for example one having the oncolytic properties based on its selective viral replication in malignant cells, and the second having a silencer expression cassette that prevents tumor growth or metastasis.

In one embodiment the method further comprises that engineered parvoviruses are administered subsequently, in case a prior virus applied renders ineffective following the host developing antibodies.

The novel oncolytic parvoviruses of this disclosure can keep an engineered shRNA expression cassette stably integrated into their genomes, while maintaining their ability to replicate, and exert oncolytic activity. Owing to their ability to express shRNAs, these novel viruses possess an additional mechanism of enhanced and synergistic action to fight cancer and are therefore anticipated to have improved anticancer properties.

As used herein, “oncolytic virus” (OV) refers to a type of virus that can infect, replicate in, and can lyse cancer cells but not normal cells. Oncolytic viruses can occur naturally or can be made in the laboratory by genetic modification of other viruses (e.g. human pathogens). Examples of oncolytic viruses include: parvovirus, Newcastle disease virus (NDV), reovirus (RV), myxoma virus (MYXV), measles virus (MV), herpes simplex virus (HSV), vaccinia virus (VV), vesicular stomatitis virus (VSV), adenovirus (AdV) and poliovirus (PV).

As used herein, “protoparvovirus” or “parvovirus” refers to rodentprotoparvovirus, a family of linear, nonsegmented, single-stranded DNA viruses, with an average genome size of 5-6 kilo base pairs (kbp). There are currently more than 100 species in the family, divided among 23 genera in three subfamilies. Parvoviruses are among the smallest viruses and are 23-28 nm in diameter. In this invention we refer—in particular—to the protoparvovirus genus of the parvoviridae family, which includes rodent parvoviruses such as rat H-1PV, LuIII, minute virus of mice (MVM) and mouse parvovirus (MPV). These viruses are evaluated as anticancer agents. The viral capsid of a parvovirus is made up of 60 copies of two or more size variants of a single protein sequence, called VP1, VP2, etc. Inside the capsid is a linear, single-stranded DNA genome in the size range 5 kbp, so the small genome of parvovirus can encode only a few proteins. At the 5′ and 3′ ends of this genome are palindromic sequences that are essential for viral genome replication mechanism called rolling-hairpin replication.

As used herein, “palindromic terminal sequence” refers to the distal sequence of inverted terminal repeats found in the viral genome. The repeats are palindromic, allowing a hairpin to form for initiation of viral genome synthesis.

As used herein, “non-coding region” refers to a segment of the viral genome that does not comprise a known viral gene, thus does not encode a known viral protein. The function of this region for the virus life cycle remains to be characterized (see below).

As used herein, “wild type” refers to a natural, unmutated, un-engineered form of genome.

As used herein, “shRNA” or “short hairpin RNA” refers to a short sequence of RNA that makes a tight hairpin turn and can be used to silence gene expression via RNA interference (RNAi). Expression of shRNA can be accomplished by delivery of plasmid or through viral or bacterial vectors into a host cell.

As used herein, a “shRNA expression cassette” refers to a distinct component of vector DNA including a shRNA sequence consisting of a sense and antisense sequences separated by a loop sequence and a promoter sequence which regulates the expression of the shRNA, for instance a RNA polymerase (pol) III promoter like the human H1 pol III promoter.

As used herein, “restriction site” or “restriction recognition site” refers to the locus on a DNA molecule where a restriction enzyme recognizes and acts. Restriction sites are typically 4-8 base pairs in length with a specific nucleotide sequence.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| GBM | glioblastoma |
| LP | Left palindromic terminal sequence |
| mAb | Monoclonal antibody |
| MPV | Mouse parvovirus |
| MVM | Minute virus of mice |
| NS | Non-structural proteins |
| OV | Oncolytic virus |
| pfu | Plaque forming unit |
| PV | Parvovirus |
| RP | Right palindromic terminal sequence |
| TME | Tumor microenvironment |
| VP | Viral capsid protein |
| Vg | Viral genome |

### BRIEF DESCRIPTION OF THE DRAWINGS

FIG. 1A. Schematic view of first generation of H-1PV-silencer vectors and their parental plasmids from which were generated. First generation of H-1PV-silencer are based on H-1PV and AH-1PV (containing a deletion in position 2022-2135) genomes. The genome contains two promoters P4 and P38 driving the expression of nonstructural (NS) and viral particle (VP) gene units, respectively. At the extremities of the genome there are left (LP) and right (RP) palindromic sequences required for virus DNA replication and packaging. The NS transcription unit encodes the NS1 and NS2 proteins, while the VP unit the VP1 and VP2 capsid proteins plus the non-structural Sat protein (not indicated in the draw). A shRNA expression cassette in which the shRNA expression is under a H1 RNA Pol III promoter, was inserted into the non-coding region of these viral genomes at the HpaI enzyme restriction site.

FIG. 1B. Schematic representation of PV production protocol used to analyze cassette stability into the PV genome. PVs are generally produced following a two steps protocol. In a first step, the plasmid harboring the viral genome is transfected into HEK293T cells. After 3-4 days from plasmid transfection, cytopathic effects are observed as a result of virus production. Cells are then harvested and lysed via three successive freeze-thaw cycles in which progeny viral particles are released in the cell lysates. The cell lysate containing virus particles is then used as inoculum for the production in NB324K producer cell line. In this specific example, three amplifications cycles (passages) were carried out in which virus produced at the end of a cycle was used as inoculum for the infection of fresh cells. At the end of each passage, virus particles were isolated, viral DNA extracted and quantified by qPCR according to the protocol described in the Materials and Methods.

FIG. 1C. Electrophoresis photo showing the shRNA expression cassette is lost during ΔH-1PV-silencer propagation. To investigate cassette stability into ΔH-1PV-sil-shEGFP viruses, after a first round in HEK-293T cells in which viruses were produced via viral DNA transfection (T), three consecutive infection rounds were carried out in NB324K cells (P1, P2, and P3) as described in panel B. At the end of each passage viral DNA was isolated and the genomic stability of the shRNA expression cassette was determined by PCR using primers laying outside the cassette. Note that the presence of the shRNA expression cassette into the viral genome, generates a larger PCR fragment than that obtained using as a template the DNA of the parental virus (ΔH-1PV). After propagation in cell culture, the PCR fragment becomes smaller suggesting that ΔH-1PV-silencer loses its cassette.

FIG. 1D. Sequence comparison between wildtype H-1PV, ΔH-1PV and ΔΔH-1PV-v1, showing that the loss of the shRNA expression cassette is associated with a 35 nt deletion within the untranslated region of parvovirus genome. DNA sequencing demonstrated that ΔH-1PV-sil-shEGFP loses the shRNA expression cassette during propagation. Together with the cassette, other deletions are generated in the non-coding region of H-1PV. The genome of one of these mutants ΔΔH-1PV-v1 is shown. This mutant features a deletion of 35 nucleotides into its untranslated region

FIG. 2A. Schematic view of H-1PV-supersilencer. H-1PV-supersilencer was constructed by inserting the shRNA expression cassette including a shRNA against EGFP into the untranslated region of ΔΔH-1PV-v1 (FIG. 1D).

FIG. 2B. Electrophoresis photo showing the shRNA expression cassette remains stably integrated into H-1PV-supersil-shEGFP. Viruses produced in HEK293T cells, were used as an inoculum for the infection of NB324K cells. After each passage, virus particles were purified, titrated and used to infect a new, freshly seeded cells according to the scheme depicted in FIG. 1B. Cells were infected with viruses used at a MOI of 100 Vg/cell. For H-1PV-supersil-shEGFP each passage was 6-8 days long while for ΔH-1PV-sil-shEGFP was 3-4 days. For each passage, virus DNA was extracted and the genomic fragment supposed to contain the shRNA expression cassette was amplified by PCR. In H-1PV-sil-shEGFP virus the cassette started to be degraded after P2 while in H-1PV-supersil-shEGFP the cassette remained stably integrated for all the three passages.

FIG. 2C. Comparison between H-1PV-super silencer and ΔH-1PV-sil, showing that the shRNA expression cassette remains stably integrated into H-1PV-super silencer for a higher number of virus amplification rounds than ΔH-1PV-sil. Virus particles used as inoculum and those produced at the end of each amplification cycle (passage, P) were quantified by qRT-PCR with a specific parvovirus NS1 probe. Columns represent number of viral genome (Vg) produced for each passage calculated making the difference between Vg values obtained at the end of the amplification cycle and the Vg used as input divided for the number of seeded cells.

FIG. 2D. Plaque assay of ΔH-1PV-sil-shEGFP and H-1PV-sil-shEGFP. H-1PV-supersil-shEGFP maintains its ability to infect cells and replicate. Plaque assay. Plaque assay was performed as described in the Materials and Methods section after five days from infection. The arrow shows one typical plaque of many plaques present in the dish.

FIG. 2E. Crystal violet staining of U373 glioma cells and HeLa cervical carcinoma cells being infected by H-1PV-supersil-shEGFP. H-1PV-supersil-shEGFP induces cytopathic effects. The U373 glioma derived and cervical carcinoma derived HeLa cells were infected with H-1PV-supersil-shEGFP at the indicated concentrations (PFU/cell). At 4 days post infection, clear changes in the morphology of the cells were observed by light microscopy. Cells were then fixed and stained with crystal violet before photography.

FIG. 2F. MTT assay and LDH assay of the U373 glioma cells. H-1PV-supersil-shEGFP induces cell lysis. MTT and LDH assays. U373 cells were infected or not with H-1PV-supersil-shEGFP. After 3 days, cell viability was determined by MTT assay and virus induced cell lysis was determined by LDH assay as described in the Materials and Methods section.

FIG. 2G. Fluorescent microscopy showing the comparison of the silencing effect of different viruses. H-1PV-supersil-shEGFP has ability to silence gene expression. ΔH-1PV-sil-shEGFP and H-1PV-supersil-shEGFP were used for the infection of U373 glioma cells. After 20 hours, cells were super-infected with a replication deficient Adenovirus expressing EGFP (Ad-EGFP). After 4 days, cells were fixed and EGFP signal evaluated by fluorescence microscopy. A representative black and white image is shown.

FIG. 2H. Fluorescence intensity quantification of the results shown in FIG. 2G. The EGFP intensity signal was quantified using the Fiji-ImageJ software. The numbers on top of the columns represent the EGFP intensity calculated by analyzing 350 positive cells. EGFP intensity was calculated after subtraction of the background value captured in green channel images of non-infected cells.

FIG. 3A. Schematic representation of LuIII-silencer. LuIII-sil-shEGFP was constructed by inserting a shRNA expression cassette (including the H1 pol III promoter and a shRNA sequence targeting the EGFP gene), into the untranslated region of the LuIII genome at position 4575.

FIG. 3B. Electrophoresis results of LuIII and LuIII-sil-shEGFP. The shRNA expression cassette is stably integrated into the LuIII viral genome. Experiments were performed as described in FIGS. 2B and C and according to the scheme depicted in FIG. 1B. Amplification of LuIII genome was performed by PCR using primers laying outside the inserted shRNA expression cassette as described in the Materials and Methods section.

FIG. 3C. The shRNA expression cassette remains stably integrated into LuIII-sil-shEGFP. Indicated are the LuIII-sil-shEGFP particles produced after each cycle of amplification (P) normalized to the number of seeded cells. For all these rounds of amplification the cassette remained stably integrated into the LuIII genome.

FIG. 3D. Fluorescent microscopy results showing the silencing effect of different viruses. The LuIII-silencer is efficient in gene silencing. U87 cells were infected with LuIII or LuIII-sil-shEGFP. After 20 hours, cells were superinfected with Ad-EGFP used at MOI of 15 (transduction unit/cell). After 1 day, EGFP positive cells were analysed with a fluorescent microscopy as described in FIG. 2G.

FIG. 3E. The fluorescence intensity quantification of the results shown in FIG. 3D. The quantification of EGFP intensity was carried out as described in FIG. 2H.

FIG. 3F. Plaque assay of LuIII and LuIII-sil-shEGFP. LuIII-sil-shEGFP keeps its ability to infect cells and replicate in a similar way than LuIII wild type. Plaque assay was performed as described in the Materials and Methods section. Arrow shows one typical plaque.

FIG. 3G. Light microscopy of crystal violet staining of cells infected by LuIII and LuIII-sil-shEGFP. LuIII-sil-shEGFP has oncolytic activity. U373 glioma cells were infected with LuIII or LuIII-sil-shEGFP used at the indicated (PFU/cell). After 72 hours, infected cells exhibited strong cytopathic effects. Cells were fixed, stained with crystal violet and analyzed by light microscopy.

FIG. 3H. MTT assay and LDH assay of U87 glioma cells infected by LuIII and LuIII-sil-shEGFP. LuIII-silencer anticancer activity. U87 cells were infected with LuIII-sil-shEGFP at the indicated MOIs (PFU/cell). After 3 days from infection, cells were processed for MTT (cell viability) and LDH (virus induced cell lysis) assays as described in the Materials and Methods section.

FIG. 4. Western blotting results of Act-1 protein in U251 glioma cells infected by different constructs of virus. Three sequences targeting the TRAF3IP2 gene were designed and inserted into the ΔH-1PV-silencer genome. Viruses were produced and used for the infection of U251 glioblastoma derived cell line. After 96 hours, cells were harvested, total protein cellular lysates prepared and analyzed by Western blotting for the content of Act-1 protein. 3-actin was used as a loading control.

FIG. 5A. Analysis of shRNA expression cassette stability. After several rounds of amplification following the production protocol illustrated in FIG. 1B, viral DNAs from the indicated viruses were extracted and analyzed by RT-PCR using primers laying outside the inserted shRNA expression cassette as described in FIGS. 2 B and C. Electrophoresis results are shown demonstrating that the shRNA expression cassette including shRNAs against TRAF3IP2 and PD-L1 remains stably integrated into the LuIII viral genome with the exception of one construct, the LuIII-sil-shTRAF3IP2(ASO1), in which the cassette starts to be lost during the P2 amplification cycle.

FIG. 5B. Sequence comparison between wildtype LuIII and ALuIII. DNA sequencing demonstrated that LuIII-sil-TRAF3IP2(ASO1) loses the shRNA expression cassette during long propagation. Together with the cassette, other deletions are generated in the non-coding region of LuIII. The genome of one of these new variants (ALuIII) is shown. This mutant together with the cassette, lost 16 nucleotides corresponding to nt 4564-4579 of the LuIII wild type genome (NCBI reference sequence M81888.1).

FIG. 5C Analysis of shRNA expression cassette stability. The shRNA expression cassette was amplified from extracted viral DNA by RT-PCR using the protocol described in FIG. 5A. Electrophoresis results are shown demonstrating that the shRNA expression cassette ASO1, previously unstable into the LuIII wt genome (FIG. 5A) instead remains stably integrated into the ALuIII viral genome at the P2 amplification cycle.

FIG. 5D. Plaque assays. Indicated purified viruses were assayed for their ability to form plaques. Plaque assays in NB324K producer cells were performed five days after infection as shown in FIGS. 2D and 3F. The arrows show typical plaques among many plaques present in the dish. The formation of plaques demonstrates the ability of the LuIII silencer to replicate efficiently and be fully infectious irrespective of the shRNA sequence inserted.

FIG. 5E. Analysis of Act-1 protein levels in total cell lysates from U251 and SNB-19 glioma cell lines. Two distinct shRNA sequences targeting different exons of the TRAF3IP2 gene were designed and inserted into the LuIII-silencer genome. Indicated viruses were produced, purified and used for the infection of U251 or SNB19 glioblastoma derived cell lines at a concentration of MOI 10 PFU/cell. After 72 hours, cells were harvested, total protein cellular lysates prepared and analyzed by Western blotting for the content of Act-1 protein. 3-actin was used as a loading control.

FIG. 5F. Analysis of PD-L1 protein levels in total cell lysates from U251 glioma derived cell line. Indicated viruses were produced, purified and used for the infection of U251 glioblastoma derived cell line at a concentration of MOIs 1, 10 and 50 PFU/cell. After 96 hours, cells were harvested, total protein cellular lysates prepared and analyzed by Western blotting for the content of PD-L1 protein. 3-actin was used as a loading control.

FIG. 6 A-B. Insertion of shRNA sequences targeting the TRAF3IP2 gene into LuIII silencer enhances its antiproliferative and oncolytic activities. MTT (cell viability) and/or LDH (virus induced cell lysis) assays were performed in U373 glioma (A) and in PC3 derived prostate cancer cells (B). Cells were seeded in 96 wells plates and then infected with the indicated viruses used at MOI 50 (U373) or MOI 10 (PC3) PFU/cell. After 3 (U373) or 4 (PC3) days from infection, cells were processed for MTT and/or LDH assays as described in FIGS. 2F and 3H.

FIG. 6C. Insertion of the shRNA expression cassette into the LuIII genome does not increase significantly unwanted cytotoxicity in normal astrocytes. Normal human astrocytes were seeded in 96 well plates in astrocytes medium (described in Materials and Methods) and grown for 72 hours before to be infected with the indicated viruses at a concentration of MOIs 10 and 25 PFU/cell. After 3 days from infection, cells were processed for LDH assay as described in FIGS. 2F and 3H.

FIG. 7. Neutralizing antibodies raised against H-1PV do not block the infection of LuIII and vice versa. NB324K cells were pretreated or not with specific neutralizing antibodies either anti H-1PV or LuIII. After 2 hours, cells were infected with H-1PV or LuIII viruses at MOI 50 PFU/cell or left untreated. After additional 20 hours, cells were fixed, stained with anti-parvovirus NS1 antibody and NS1 positive cells evaluated by fluorescence microscopy. Representative black and white images are shown.

FIG. 8A-K. Synthesized DNA fragments used for cloning.

### DETAILED DESCRIPTION

The disclosure provides novel oncolytic protoparvoviruses with the ability to express shRNAs for silencing target genes that can be used to treat cancer. The novel oncolytic protoparvoviruses have increased cargo capacity, as well as increased capacity of keeping a heterologous sequence-such as a silencing cassette-stably integrated into their genome after several passages of virus amplification and propagation.

Glioblastoma (GBM) is the most aggressive and common type of primary malignant brain tumor in the adult brain. GBM remains uniformly fatal with dismal median overall survival of only 12-15 months and with only 4.5% of patients surviving more than 5 years. Hence, new therapeutic options are urgently needed. Recent advances in rescuing the anticancer immune response by vaccination with tumor-specific peptides and/or by anti-checkpoint blockers have reinvigorated the enthusiasm for the immunological approach to fight cancer with some impressive results obtained in the treatment of certain tumor types (e.g. melanoma, prostate cancer). However only a fraction of patients responds to treatment and for brain tumors, results obtained so far indicate even less success as exemplified by a Phase 3 ICI study (Checkmate-143) which failed to show a survival benefit in recurrent GBM patients treated with nivolumab (anti-PD1 mAb).

Oncolytic viruses (OVs) have the capacity to selectively infect and destroy cancer cells without harming normal cells. Besides, OVs can stimulate strong anti-tumor immune response and destroy tumor vasculature. As a result, not less than forty OVs are currently evaluated in clinical trials against a variety of different cancers as a monotherapy or in combination with other anticancer agents.

Among the oncolytic viruses, H-1PV is a candidate under research. H-1PV belongs to the parvoviridae family, genus protoparvovirus (PV). The genus includes other rodent PVs, such as the minute virus of mice (MVM), the mouse parvovirus (MPV) and LuIII. The natural host of H-1PV is the rat, thus it is not a human pathogen to trigger pre-existing immunity. This represents an advantage of H-1PV over other OVs based on human pathogens (e.g. HSV and Ad), as H-1PV may have a larger therapeutic window before the occurrence of neutralizing antibodies.

H-1PV has an icosahedral capsid which contains a linear, single stranded DNA genome of about 5100 nucleotides. The genome includes the P4 and P38 promoters, which regulate the expression of the NS and VP transcription units, respectively. The NS unit encodes the non-structural 1 (NS1) and NS2 proteins while the VP unit encodes for the VP1 and VP2 capsid proteins and for the non-structural Sat protein. NS1 is the major effector for virus oncotoxicity. At its right and left ends, the genome presents terminal palindrome sequences that are required for viral DNA replication and encapsidation. In addition, between VP2 stop codon and the right-hand palindromic sequence there is a non-coding region, whose functions remain largely unknow.

H-1PV can efficiently infect abroad range of human cancer cell lines from different origins, and has been shown to display oncolytic and oncosuppression properties. First phase I/IIa clinical study of H-1PV monotherapy in glioma patients demonstrated the safety and tolerability of the treatment. Furthermore, the clinical study shows that virus treatment is associated with first signs of anticancer efficacy including tumour microenvironment immunoconversion and improvement of median progression free survival and overall survival in comparison with historical controls. However, at the regimes used, the virus was unable to eradicate the tumor by itself. H-1PV has the following characteristics that are suitable for tumor treatment: (i) non pathogenicity, excellent tolerance and possible efficacy in humans for treating patients with malignant brain tumors and other solid tumours; (ii) lack of pre-existing antiviral immunity in the human population; (iii) natural oncotropic and oncosuppressive properties, and (iv) anti-cancer immunostimulating activities without the need to modify the virus.

While it is possible to improve anticancer efficacy of other oncolytic viruses by inserting a therapeutic gene into the viral genome and providing the virus with a new complementary mechanism of action to kill cancer cells and/or to stimulate the immune system to act against the cancer more efficiently, the limited payload capacity of H-1PV hampers this kind of modifications. Only small foreign sequences (200-250 nucleotides) can be inserted in H-1PV genome without affecting the ability of the virus to self-replicate, and many of the therapeutic transgenes used in cancer gene therapy are too large to be inserted into the H-1PV genome.

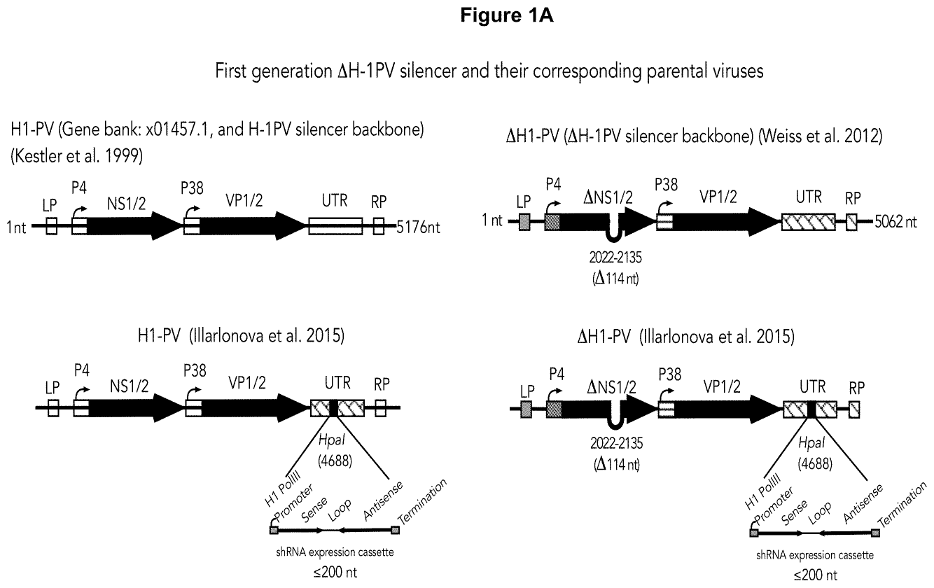

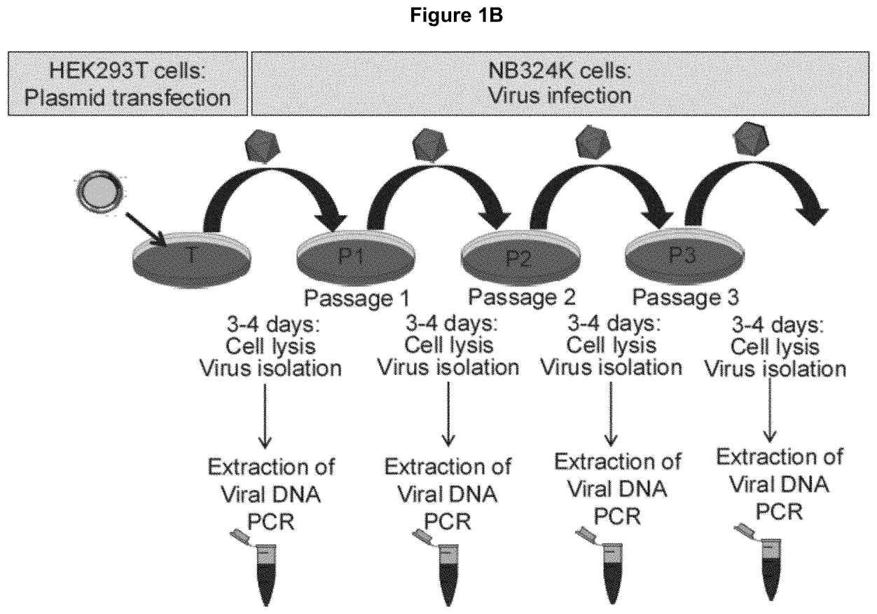

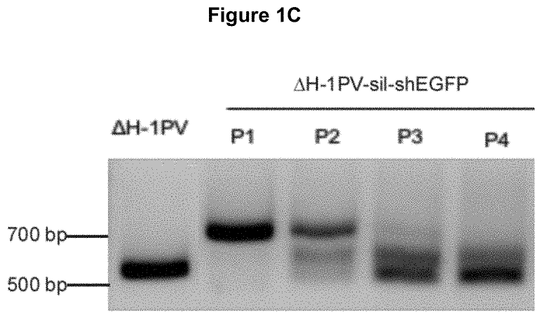

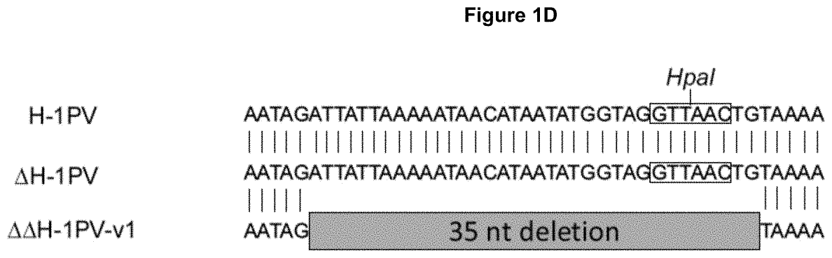

Despite these limitations, inserting regulatory elements has been previously achieved by the inventors. For example, as shown in FIG. 1A, a shRNA expression cassette (consisting of a shRNA and a H1 RNA PolIII promoter driving its expression) has been inserted into the non-coding region of the H-1PV genome at the HpaI enzyme restriction site using as a backbone the plasmid. The resulting virus, H-1PV-silencer, expresses shRNA at a high level and is capable of silencing target genes, such as oncogenes.

FIG. 1A also shows ΔH-1PV-silencer, which has a deletion at the position 2022-2135 of the viral genome at the C-terminus region of parvoviral NS2 coding region, in addition to the shRNA expression cassette insertion. This deletion was originally found in a naturally-occurring H-1PV variant and it was described to enhance the efficiency of the virus.

Additionally, the inventors also inserted a shRNA against CDK9, a gene whose activity is often dysregulated in cancer, reinforced the oncolytic activity of H-1PV against pancreatic and prostate derived cancer cell lines in comparison with wild type virus. By comparing the results against xenograft nude rat models of human pancreatic (AsPC-1) and prostate (PC3) carcinomas, it is confirmed that (Δ)H-1PV-sil-shCDK9 has enhanced oncosupressive activity than control viruses (parental and virus expressing shRNAs against EGFP). The results show a significant increase of the overall survival of ΔH-1PV-sil-shCDK9 treated animals.

However, despite these substantial improvements, by further characterizing (Δ)H-1PV-silencer, the inventors discovered that the shRNA expression cassette does not remain stably integrated into the viral genome but on the contrary is gradually (partially or even entirely) lost during the propagation of the virus in the NB324K production cell line (data not shown). In view of a therapeutic use of (Δ)H-1PV-silencer, and in particular considering the manufacturing of clinical-grade virus batches, that requires several rounds of virus amplification, it is desirable that the shRNA expression cassette is stably kept into the viral genome and the virus produced maintains its original properties.

Therefore, a novel solution that overcomes the problem of instability of the shRNA expression cassette inserted into the parvovirus genome is described herein.

The brain tumor microenvironment (TME) modulates the interaction and communication between the host and tumor in favor of tumor growth. The TME plays a crucial role in GBM development, dissemination and resistance. Indeed, the resistance of GBM stem-like cells to radiotherapy and chemotherapy may arise from their interactions with the GBM TME. In general, TME components enhance tumor-related inflammation with the production of immune-inhibitory cytokines that foster immune-tolerance and avoids immune rejection. Inflammation in the TME is crucial in GBM pathology, as it regulates the directional movement of immune cells and stem cells. For instance, immunomodulatory cytokines stimulate macrophage differentiation toward immunosuppressive M2 phenotype which secrete IL-10. Secretion of IL-10 by M2 macrophages inhibits interferon gamma production and downregulates MHC class I receptors on antigen presenting cells (APC).

The NF-κB and JNK/AP-1 pathways play crucial roles in inflammation in GBM and promote the expression of inflammatory cytokines, including IL-8, IL-1, IL-6 and granulocytes macrophage colony stimulating factor (GM-CSF). Furthermore, inflammation in the TME facilitates tumour development directly by enhancing stromal involvement, causing angiogenesis and tumour progression.

Therefore, development of a genetically modified parvoviruses with enhanced anticancer activity, e.g. PVs expressing shRNA targeting TRAF3IP2, may provide a key to the success of PV in clinical setting.

The following materials and methods are used.

Cell Culture

NB324K human newborn kidney cells transformed with simian virus 40 (SV40) have been previously described13. HEK293T cells has been obtained from ATCC (LGS Standards GmBH, Wesel, Germany). Prostate cancer derived PC3 cell line and Glioblastoma derived U251 and SNB19 cell lines were obtained from National Cancer Institute (Rockville, Md., USA). Glioblastoma derived U373, and U87 cell lines were obtained from Dr. Iris Augustin (DKFZ, Heidelberg, Germany), and the cervical carcinoma derived HeLa cell line (DKFZ, Heidelberg, Germany [28]). Astrocytes cells were purchased from ScienCell research laboratories (Carlsbad, Calif., USA). All glioma cell lines were grown in Dulbecco's Modified Eagle Medium (DMEM) (Sigma-Aldrich, Munich, Germany). PC3 cell line was grown in RPMI-1640 medium (Sigma-Aldrich, Munich, Germany), NB324K cell line was grown in Minimum Essential Medium (MEM, Sigma-Aldrich, St. Louis, Mo., USA) and astrocytes were grown in astrocyte medium containing basal medium supplemented with astrocyte growth supplement (ScienCell research laboratories). All media were supplemented with 2% (for astrocyte medium) or 5% (for MEM) or 10% (for all other cell culture medium) Fetal Bovine Serum, 100 units/ml penicillin, and 100 μg/ml streptomycin (all from Gibco, Karlsruhe, Germany). All cells were grown at 37° C. in 5% CO2, 95% humidity and routinely checked for mycoplasma contamination.

Plasmid Construction

The infectious molecular plasmid pDelH1, containing the ΔH-1PV genome variant featuring a deletion in the nucleotides 2022-2135 (NS-coding region) has been previously described (Weiss, Stroh-Dege et al., 2012). The pDelH1-based infectious molecular plasmids, pΔH-1PV-silencer (pΔH-1PV-sil) which contains a shRNA expression cassette (whose shRNA expression is under the control of on H1 pol III promoter) and pΔH-1PV-sil-shEGFP (a pΔH-1PV-silencer expressing a shRNA targeting the EGFP gene) have been also previously described. The pDelH1 was used as a backbone to introduce the additional 35 nucleotides deletion [nucleotides (nt) 4659-4693] into the untranslated region of the ΔH-1PV genome (nt numbers are according to the H-1PV genome NCBI reference sequence X01457.1) using a DNA synthesis approach (see below). The deletion was found by DNA sequencing after propagation of the ΔH-1PV-sil-shEGFP virus into NB324K cells and PCR-amplification of H-1PV genome fragment from nt 4280 till nt 4846 using the following primers Forward: 5′-AAAACAACCCACCAGGTCAA-3′ (corresponding to nt 4280-4299 of H-1PV genome) and Reverse: 5′-GTACCAACCAACCACCCAAC-3′ (nt 4865-4846). The PCR product was cloned into pCR-Blunt II-TOPO vector (Thermo Fisher Scientific, Carlsbad, Calif., USA), transformed in Escherichia coli strain SURE (Invitrogen, Darmstadt, Germany) and subjected to DNA sequencing. One of the sequenced clones (ΔΔH-1PV-v1) displayed the 35 nt deletion.

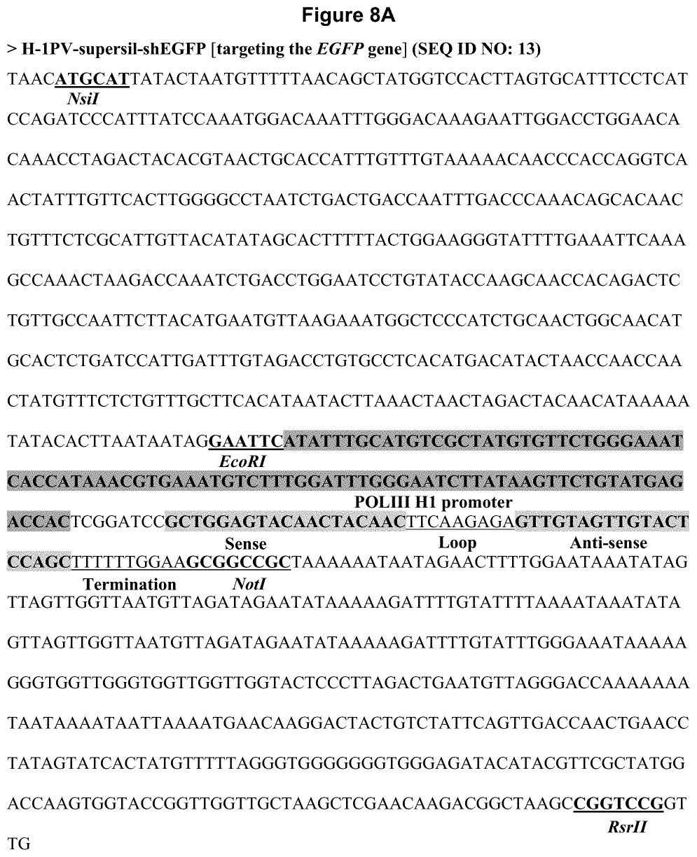

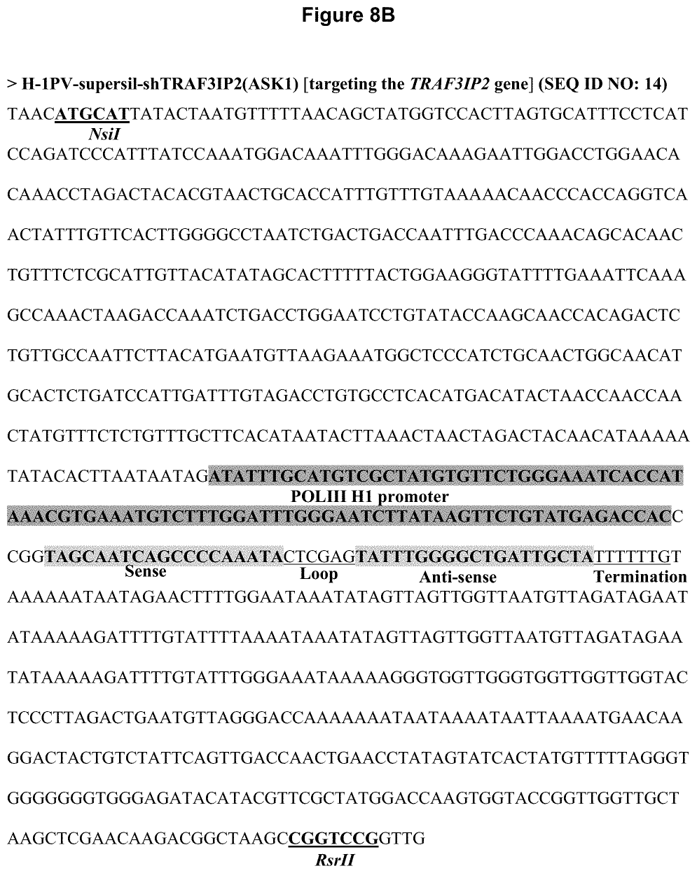

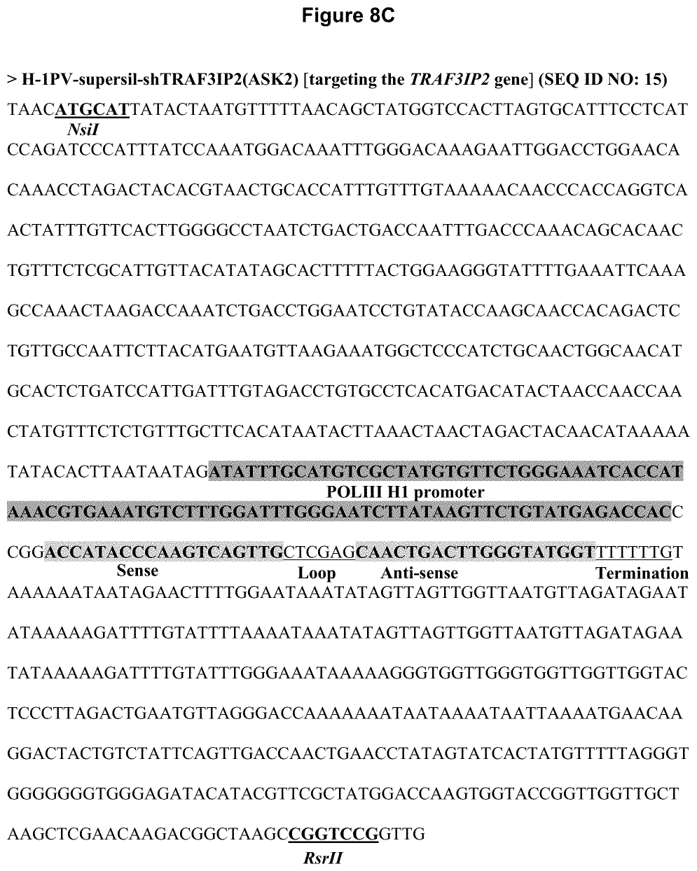

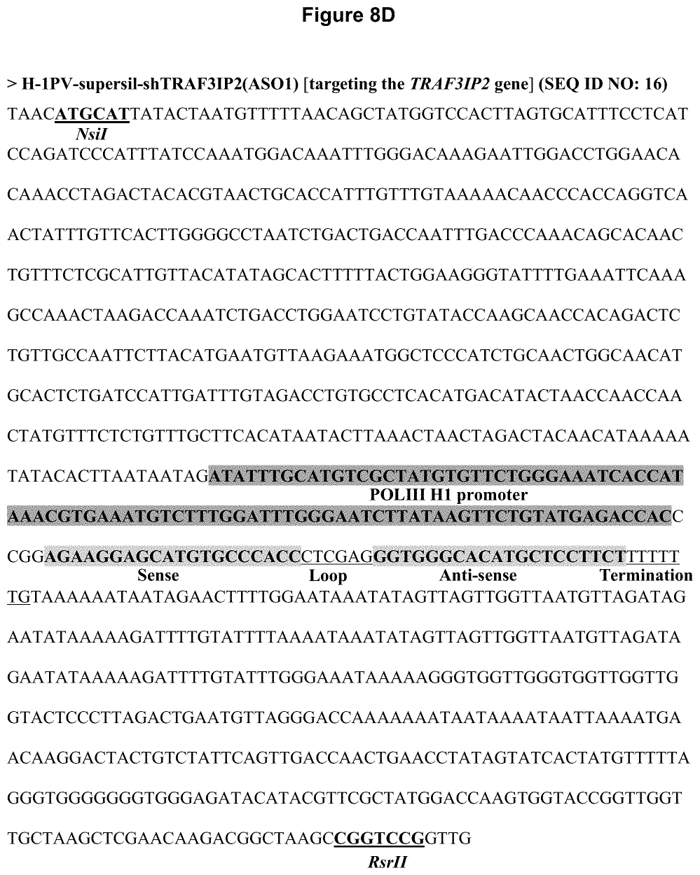

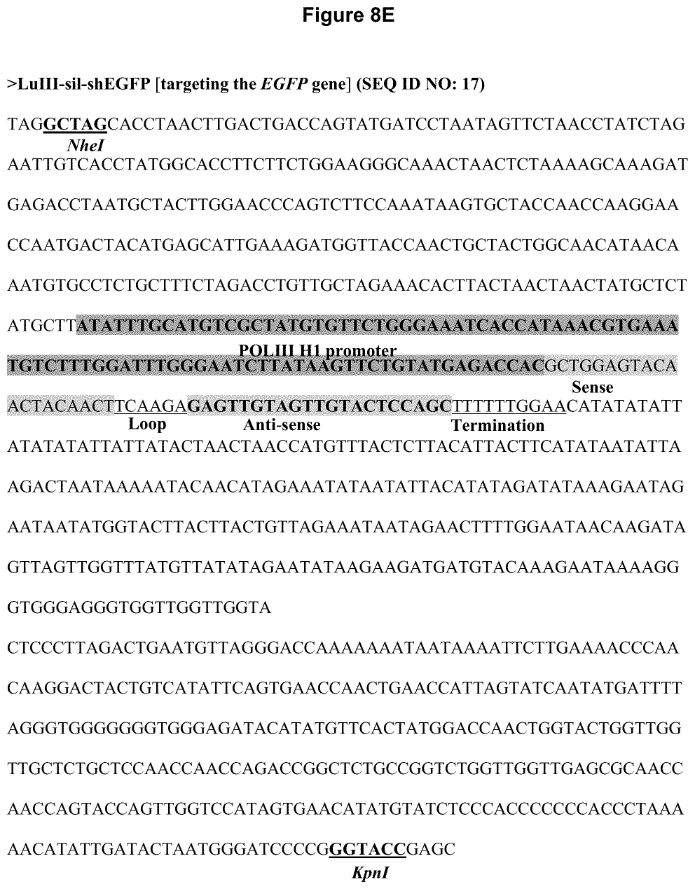

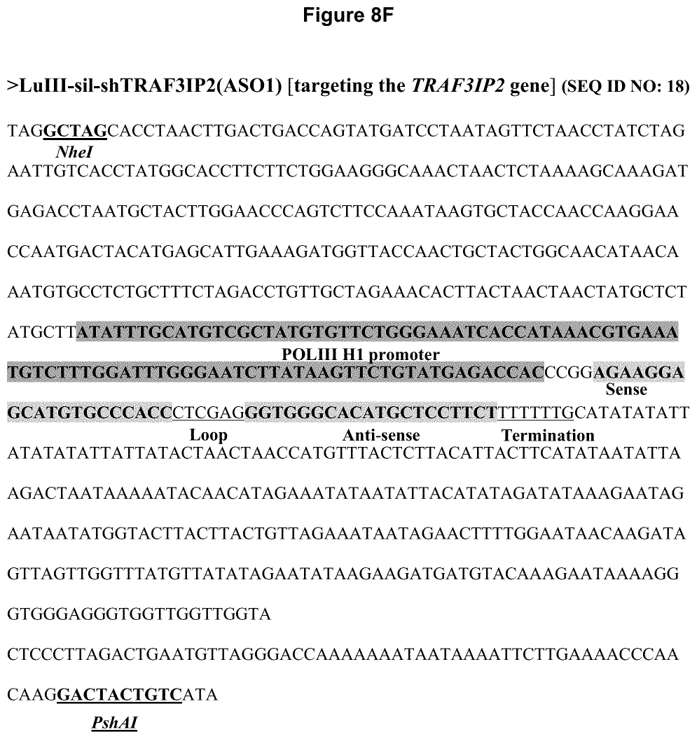

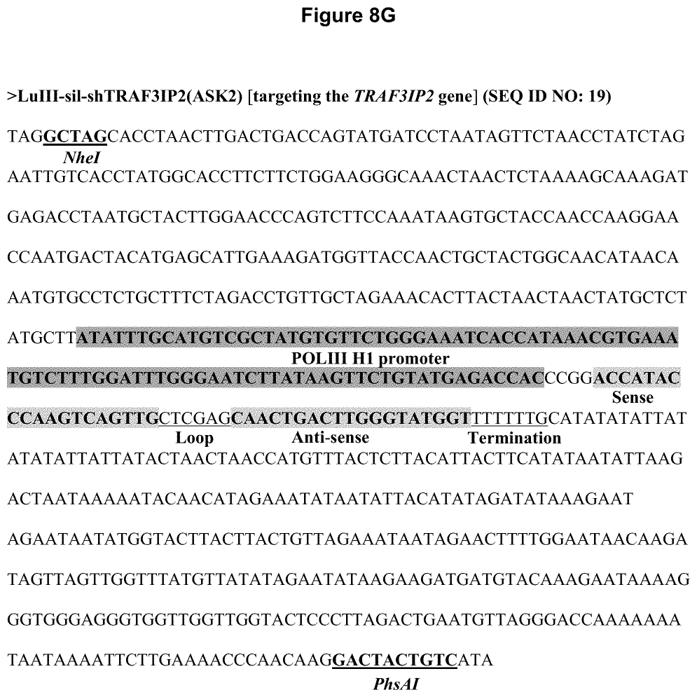

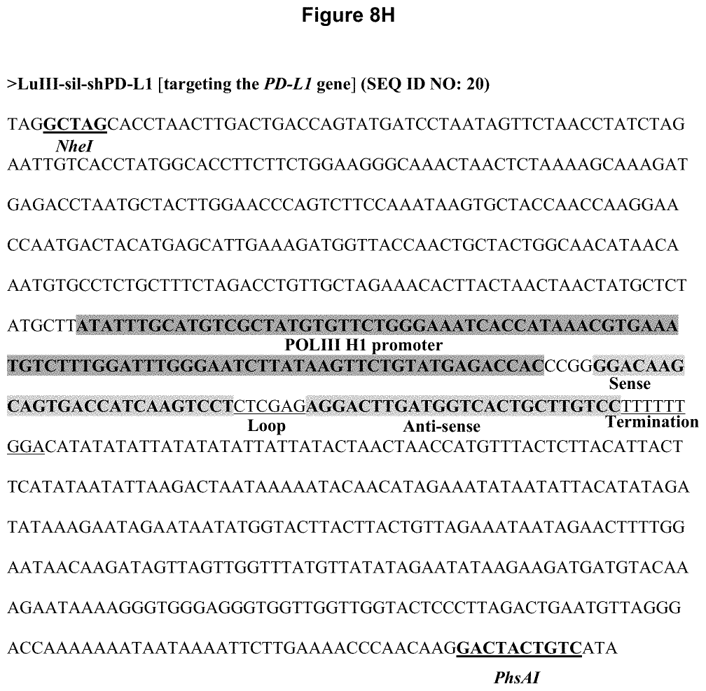

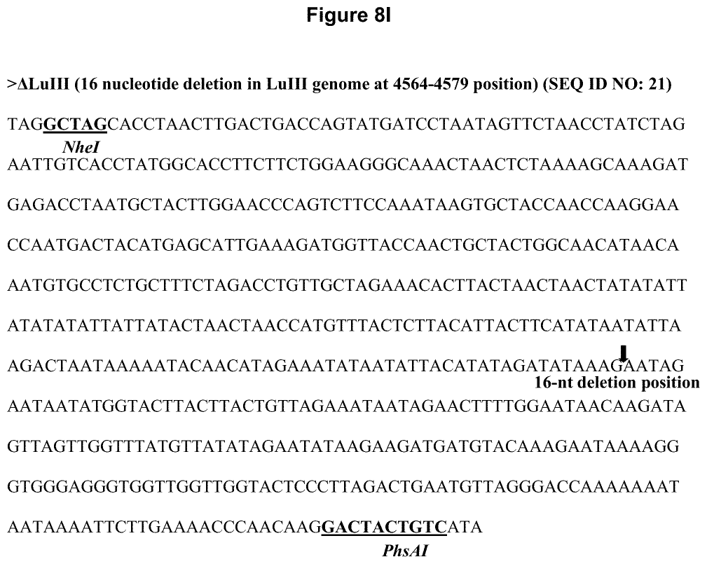

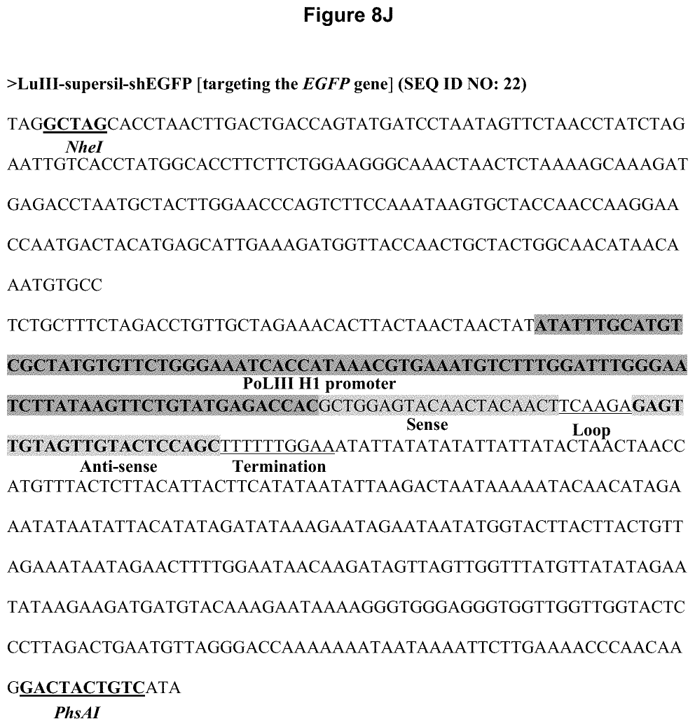

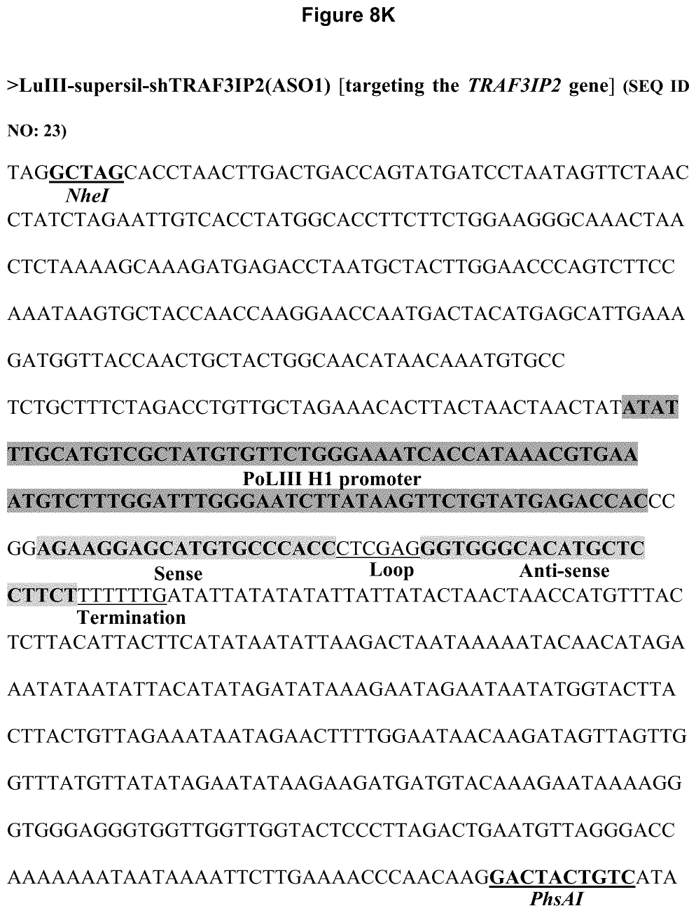

A DNA fragment analogous to the H-1PV genome region comprised between the NsiI and RsrII restriction enzyme sites (nt 4132-5065, NCBI reference sequence X01457.1) featuring the 35-nt deletion was synthesized by GENEWIZ (South Plainfield, N.J., USA) and cloned into the NsiI and RsrII digested pDelH1 plasmid thus generating pΔΔH-1PV-v1 (see FIG. 8 for the synthesized DNA fragments used for the cloning).

pH-1PV-supersilencer plasmids containing the same shRNA expression cassette as pΔH-1PV-silencer and shRNAs sequences targeting EGFP or TRAF3IP2 were constructed using the same cloning strategy (table 1 shows the inserted shRNA sequences). The shRNA targeting the EGFP and TRAF3IP2 genes (ASO1, specific against exons 9 and 10) have been previously described (14). ASK1 and ASK2 targeting the TRAF3IP2 gene, on exon 8 and 3 respectively, were de novo synthesized using the oligo synthesis service of GENEWIZ.

The infectious molecular plasmid pLuIII containing the LuIII genome has been previously described. For the insertion of the specific shRNA expression cassettes into the LuIII viral genome, specific DNA fragments including part of the viral genome and the shRNA expression cassette were synthesized by GENEWIZ (South Plainfield, N.J., USA) and cloned into pLuIII. These clones differ only for the shRNAs sequences (listed in Table 1). In particular, a DNA fragment including the NheI restriction enzyme and nucleotides 4288-5135 of the LuIII genome (NCBI reference sequence M81888.1) and part of vector backbone until the KpnI site (outside of the LuIII genome) in which the shRNAs expression cassette containing a shRNA targeting the EGFP gene was inserted at position 4575 (NCBI reference sequence M81888.1) was synthesized and cloned into NheI and KpnI digested pLuIII plasmid, thus generating pLuIII-sil-shEGFP. For the cloning of LuIII plasmids including the shRNA sequences targeting the TRAF3IP2 or PD-L1 genes (Table 1), DNA fragments encompassing the region between NheI and PhsAI restriction enzymes and containing the shRNA expression cassette inserted into the same position previously used for pLuIII-sil-shEGFP (4575), were cloned into NheI and PhsAI digested pLuIII plasmid, thus generating pLuIII-sil-shTRAF3IP2(ASO1), pLuIII-sil-shTRAF3IP2(ASK2), pLuIII-sil-shPD-L1 respectively (see FIG. 8). For the pLuIII super silencer clones, a DNA fragment corresponding to the LuIII genome region comprised between the NheI and PshAI restriction enzyme sites (nt 4288-4903) (NCBI reference sequence M81888.1) but featuring the novel 16-nt deletion (nt 4564-4579) was synthesized by GENEWIZ and cloned into the NheI and PshAI digested pLuIII plasmid thus generating pALuIII. pALuIII plasmid then was used as a backbone to clone the shRNAs expression cassette sequences targeting EGFP or TRAF3IP2(ASO1) previously cloned in pLuIII. However, due to the deletion, the shRNAs expression cassettes were inserted at position 4564 (see FIG. 8).

TABLE 1
shRNA sequences

| | | | | | | Target | Targeted |
|---|---|---|---|---|---|---|---|
| No. | Name | Sense | Loop | Anti-sense | Termination | exon | gene |
| 1 | ASK1 | TAGCAATCAGC | CTCGAG | TATTTGGGGCT | TTTTTTG | 8 | TRAF3IP2 |
| | | CCCAAATA | | GATTGCTA | | | |
| | | (SEQ ID NO: 1) | | (SEQ ID NO: 2) | | | |
| 2 | ASK2 | ACCATACCCAA | CTCGAG | CAACTGACTTG | TTTTTTG | 3 | TRAF3IP2 |
| | | GTCGTTG | | GGTATGGT | | | |
| | | (SEQ ID NO: 3) | | (SEQ ID NO: 4) | | | |
| 3 | ASO1 | AGAAGGAGCAT | CTCGAG | GGTGGGCACAT | TTTTTTG | 9&10 | TRAF3IP2 |
| | | GTGCCCACC | | GCTCCTTCT | | | (Alt et al.) |
| | | (SEQ ID NO: 5) | | (SEQ ID NO: 6) | | | |
| 4 | PD-L1 | GGACAAGCAGTG | CTCGAG | AGGACTTGATGG | TTTTTTGG | 2 | PD-L1 |
| | | ACCATCAAGTCCT | | TCACTGCTTGTCC | A | | |
| | | (SEQ ID NO: 7) | | (SEQ ID NO: 8) | | | |
| 5 | EGFP | GCTGGAGTACA | TTCAAGAG | GTTGTAGTTGT | TTTTTTGG | — | EGFP |
| | | ACTACAAC | A | ACTCCAGC | AA | | |
| | | (SEQ ID NO: 9) | | (SEQ ID NO: 10) | | | |

Virus Production

All viruses were first produced in HEK293T via plasmid transfection and then amplified in NB324K cells via virus infection as previously described. Briefly, 2×10⁶ HEK293T cells were cultivated in 10 cm Petri dish and transiently transfected with 10 g/plate of viral plasmid using Lipofectamine LTX with Plus reagent (Thermo Fisher Scientific) at the plasmid to reagent ratio of 1:4 according to manufacturer's instructions. After 2-3 days, cells were harvested within their medium and lysed by 3 freeze-and-thaw cycles and cellular debris was removed by centrifugation. Produced viruses were further amplified by infecting NB324K cells (100-1000 virus genome per seeded cells). Infected cells were harvested after 3-4 [ΔH-1PV, ΔH-1PV-sil-shEGFP, LuIII, LuIII-sil-shEGFP, LuIII-sil-shTRAF3IP2(ASO1), LuIII-sil-shTRAF3TP2(ASK2), LuIII-sil-shPD-L1, ΔLuIII, LuIII-supersil-shEGFP, LuIII-supersil-shTRAF3IP2(ASO1)] or 5-8 days (H-1PVsupersil-shEGFP) post-infection. Viruses were extracted from the cells using Virus Tris/EDTA (VTE) lysis buffer pH 8.7 containing 0.05M Tris-HCl, and 0.5 mM EDTA.

Virus Titration

An aliquot of the crude virus cell lysate was used for the virus titration. Crude virus cell lysates were digested with 50 U/ml of benzonase nuclease (Sigma-Aldrich, Steinheim, Germany) for 30 min at 37° C. to digest free viral genomic DNA. The encapsided viral DNA was extracted using QiAmp MiniElute Virus Spin Kit (Qiagen, Hilden, Germany) according to manufacturer's instruction manual. Viral titration was performed by qPCR and plaque assay according to El-Andaloussi et al. and expressed either as viral genome (Vg) or plaque-forming unit (PFU) per ml.

Evaluation of the shRNA Expression Cassette Maintenance within the Parvovirus Genome

Plasmid carrying the viral genomes were first transiently transfected in HEK-293T. After 3-8 days, when cell lysis was evident, virus particles produced were isolated and quantified by PCR as described above. 100 Vg of each virus/cell was used for the infection of NB324K producer cell line. At the end of each amplification cycle, 3-4 days for ΔH-1PV-sil-shEGFP, LuIII-sil-shEGFP, LuIII-sil-shTRAF3IP2(ASO1), LuIII-sil-shTRAF3IP2(ASK2), LuIII-sil-shPD-L1, ΔLuIII, LuIII-supersil-shEGFP, LuIII-supersil-shTRAF3IP2(ASO1) or 6-8 days for H-1PV-supersil-shEGFP, a fraction of the produced virus was used as inoculum (100 Vg/cell) for the infection of freshly prepared NB324K cells. The amplification of the virus was repeated three times for ΔH-1PV-sil-shEGFP, H-1PV-supersil-shEGFP, LuIII-sil-shTRAF3IP2(ASO1), LuIII-sil-shTRAF3IP2(ASK2), and LuIII-sil-shPD-L1, four times for LuIII-sil-shEGFP and two times for ΔLuIII, LuIII-supersil-shEGFP, LuIII-supersil-shTRAF3IP2(ASO1). At the end of each cycle, cells were lysed and encapsidated viral DNA purified. The presence of the shRNA expression cassette within the parvoviral genomes, was analyzed by PCR. As controls for the PCR, pDelH1, pLuIII, and pALuIII plasmids harboring the ΔH-1PV or LuIII or ΔLuIII genome respectively, were used as templates.

For ΔH-1PV-sil-shEGFP, and H-1PV-supersil-shEGFP the primers used for the PCR were those described above in the plasmid construction section. These primers were expected to generate a PCR fragment of 756 bp in ΔH-1PV-sil-shEGFP or of 721 bp in H-1PV-supersil-shEGFP or of 586 bp in ΔH-1PV. For LuIII, LuIII-sil-shEGFP, LuIII-sil-shTRAF31P2(ASO1), LuIII-sil-shTRAF3TP2(ASK2), LuIII-sil-shPD-L1, ΔLuIII, LuIII-supersil-shEGFP, LuIII-supersil-shTRAF3IP2(ASO1), PCR was performed using the following primers: LuIII FOR: 5′-AATGCTCCAGGTCAGCTTCTGG-3′ (SEQ ID NO: 11) recognizing nt 4262-4283 of LuIII genome (NCBI reference number: M81888.1), and LuIII REV: 5′-GTCCCTAACATTCAGTCTAAGGG-3′(nt 4858-4836) (SEQ ID NO: 12). In this case the PCR fragments expected were 597 bp for parental LuIII, 745 bp for LuIII-sil-shEGFP and LuIII-sil-shTRAF3IP2(ASO1), 743 bp for LuIII-sil-shTRAF3IP2(ASK2), 761 bp for LuIII-sil-shPD-L1, 581 bp for ΔLuIII, 729 bp for LuIII-supersil-shEGFP and LuIII-supersil-shTRAF3IP2(ASO1).

LDH Assay

Cancer cell lines and primary astrocytes were seeded at a density of 4,000 cells/well in 96-well plates in 50 μl of their respective culture medium supplemented with appropriate concentration of heat inactivated FBS as described in Cell Culture section of Materials and Methods. After 24 h, 50p of FBS-free medium containing the various viruses were added. Cells were incubated for 3-4 days and then subjected to lactase dehydrogenase LDH assay (CytoTox 96 nonradioactive cytotoxicity assay, Promega, Madison, Wis., USA), according to the manufacturer's instructions. In this assay, LDH activity is determined by utilizing a coupled enzymatic reaction, where LDH oxidizes lactate to pyruvate, which subsequently reacts with iodonitrotetrazodium chloride (INT) to form water soluble formazan. The absorbance of the formazan dye was measured at 492 nm using a CLARIOstar ELISA plate reader (BMG LABTECH, Ortenberg, Germany). For each condition tested, six replicates were prepared for which three were used for calculating the total lysis in the presence of detergent as previously described.

MTT Assay

For the analysis of cell viability, the metabolic activity of nicotinamide adenine dinucleotide phosphate (NADPH)-dependent cellular oxidoreductase enzymes was assessed by MTT assays, as previously described. These enzymes catalyze the reduction of 3-(4,5-dimethylthiazol-2-yl)-2,5-diphenyl-2H-tetrazolium bromide (MTT) to a soluble colored formazan dye. The absorbance of the formazan dye was measured at 595 nm by using a CLARIOstar ELISA plate reader. The viability of infected cells was expressed as the ratio of the corresponding absorbance to that of non-infected cells taken arbitrarily as 100%. The mean percentage of MTT activity was calculated from at least three replicates for each condition tested.

Crystal Violet Staining

Cell viability was also assessed by crystal violet staining. Cells were incubated with 1% aqueous crystal violet solution (V 5265, Sigma-Aldrich) at room temperature for 15 min. Cells were then rinsed with water and then analyzed by light microscopy.

Western Blotting

At 96 hours post-infection, virus-infected cells were harvested in their medium and centrifuged at 3,000 rpm×10 min at 4° C. Cell pellet was washed twice with 1× phosphate-buffered saline (PBS) and then lysed in 500 μl of lysis buffer consisting of 50 mM Tris-HCl pH 8, 200 mM NaCl, 0.5% NP-40, 1 mM DTT, 10% glycerol and a mix of protease inhibitors (Roche Diagnostics, Mannheim Germany) and kept on ice for 30 min. After centrifugation (10,000 rpm×20 min at 4° C.) the supernatant was collected and the protein amount was measured by bicinchoninic acid (BCA) based assay using Pierce™ BCA Protein Assay Kit (Thermo Fischer Scientific, Rockford, Ill., USA) according to the manufacture's manual. Total cellular extracts 60 μg were loaded and separated on Blot™ 4-12% Bis-Tris Plus gels (Thermo Fisher Scientific, Carlsbad, Calif., USA) and transferred onto nitrocellulose membrane integrated into preassembled transfer stack with Invitrogen™ iBlot™ 2 Dry Blotting System (Thermo Fisher Scientific, Kiryat Shmona, Israel). The membranes were blocked in TBS (150 mM NaCl, and 20 mM Tris, pH 7.6) with 0.1% Tween 20 and 5% nonfat dry milk for 1 hour at room temperature. The blots were incubated with the following primary antibodies overnight at 4° C.: TRAF3IP2 (rabbit polyclonal, Sigma-Aldrich, Steinheim, Germany) used at 1:2000 dilution, PD-L1 (rabbit polyclonal, Sigma-Aldrich, Steinheim, Germany) used at 1:500 dilution and β-actin (mouse monoclonal, Santa Cruz Biotechnology, Heidelberg, Germany) used at 1:5000 dilution. After membrane washing the peroxidase-conjugated goat anti-rabbit or goat anti-mouse antibody (Santa Cruz Biotechnology, Heidelberg, Germany) was added for 1 hour at room temperature. Membranes were then washed and visualized with the Supersignal West Femto maximum sensitivity kit (Thermo Fisher Scientific) or Supersignal West Atto ultimate sensitivity kit (Thermo Fisher Scientific).

Fluorescence Microscopy Analysis

U373 and U87 cells were grown in 96-well flat bottom plates (Greiner bio-one GmbH, Frickenhausen, Germany) and then infected overnight with the various protoparvoviruses indicated in the figures. Cells were then superinfected with a recombinant adenovirus (Ad) expressing EGFP (Ad-EGFP) (VectorBuilder Inc., Chicago, USA) used at a MOI of 15 transduction units/cell. After 24 (for LuIII and LuIII-sil-shEGFP) or 72 (all other viruses) hours, cells were washed once with 1×PBS, fixed with 4% paraformaldehyde (PFA) at room temperature for 10 minutes and washed again twice with PBS. Nuclei were stained with 4′,6-diamidino-2-phenylindole (DAPI) dye. The fixed cells were examined with Evos FL Auto 2 fluorescent microscope (Invitrogen by Thermo Fisher Scientific, Bothell, Wash., USA). The EGFP intensity was quantified using the Fiji-ImageJ software to determine EGFP silencing efficacy.

Neutralizing Antibody Assay

2.5×10⁴ NB324K cells were grown overnight in 96-well flat bottom plates (Greiner bio-one GmbH, Frickenhausen, Germany). Cells were pretreated (or not) with neutralizing antibody raised against H-1PV or LuIII (anti-H-1PV: H-1-42; anti-LuIII: LuIII-T56, both antibodies were kindly provided by Dr. Jurg Nuesch, German Cancer Research Center, Heidelberg, Germany) for 2 hours. Cells were then infected with the H-1PV or LuIII at MOI 50 PFU/cell or left untreated. After 20 hours, cells were washed once with 1×PBS, fixed with 4% paraformaldehyde (PFA) at room temperature for 10 minutes. Henceforth in between steps, two washing steps were carried out with 1×PBS. The blocking buffer (1×PBS containing 10% Fetal Bovine Serum and 0.1% Triton X-100) was added to cells and incubated further at room temperature for 1 hour. After washing step, cells were incubated with NS1 SP8 antibody (German Cancer Research Center, Heidelberg, Germany, Brockhaus, K et al.) at room temperature for 2 hours followed by another washing and then incubated with goat anti-rabbit IgG (H+L) Highly Cross-Adsorbed Secondary Antibody-Alexa Fluor Plus 488 antibody (Thermo Fisher Scientific, Rockford, Ill., USA) at room temperature for 1 hour. Nuclei were stained with 4′,6-diamidino-2-phenylindole (DAPI) dye. Cells were examined with Evos FL Auto 2 fluorescent microscope (Invitrogen by Thermo Fisher Scientific Bothell, Wash., USA) for NS1 positive cells. The percent of NS1 positive cells were quantified using the Fiji-ImageJ software (Schindelin et. al.) and normalized to DAPI positive cells.

### Example 1: Isolation of Novel H-1PV Variants Featuring Deletions within the Uncoding Region of their Genome

It has been reported that the integrity of the untranslated region is essential for effective virus replication. We have propagated the ΔH-1PV-sil (FIG. 1A) in NB324K packaging cell line according to the production protocol depicted in FIG. 1B. Unexpectedly, it is found that the untranslated region containing the cassette was unstable. Indeed, PCR-mediated amplification of the region produced different fragments whose size was smaller than expected, suggesting the occurrence of deletions in that region (FIG. 1C). To find out whether all or only a part of the cassette was cut out from the ΔH-1PV-sil genome, the ΔH-1PV-sil untranslated region was amplified by PCR using as a template the lysate from ΔH-1PV-sil-shEGFP infected cells and PCR primers lying outside the cassette insertional site. The PCR product was then cloned into pCR-Blunt II-TOPO plasmid and used for bacteria transformation. DNA was isolated from single colonies and subjected to DNA sequencing. It is discovered that in some clones together with all or part of the cassette, the virus was also losing some nucleotides of its genome. In particular in one of these variants (named ΔΔH-1PV-v1), the deletion was of 35 nucleotides upstream the shRNA expression cassette insertional HpaI site, corresponding to position 4659-4693 of the wild type H-1PV genome (FIG. 1D).

Here it is discovered that the inserted shRNA expression cassette in first generation H-1PV silencer vectors is prone to deletion. NB324K production cell line infected by ΔH-1PV silencer underwent different cycles of passaging. The viral genome was obtained after each passaging, and the untranslated region of the viral genome was sequenced and compared. It is discovered that the shRNA expression cassette does not remain stably integrated into the viral genome. Instead, the shRNA expression cassette is gradually lost, partially or entirely, during the propagation of the virus in the cells.

This discovery has a major implication, because to be effectively used in cancer treatment the virus needs to be able to stably replicate and express the shRNA. Therefore, improving the stability of ΔH-1PV-silencer increases its efficacy in treating cancer by providing synergistical effects.

### Example 2: ΔH-1PV-Supersilencer with Stable shRNA Expression Cassette

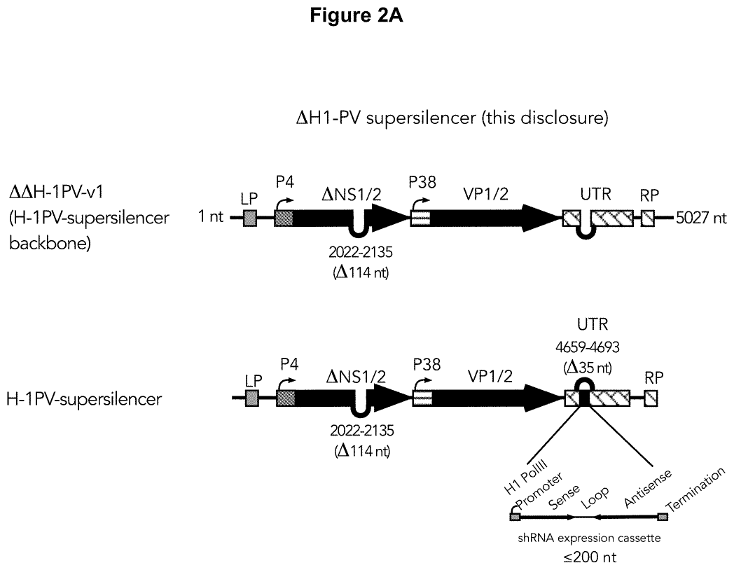

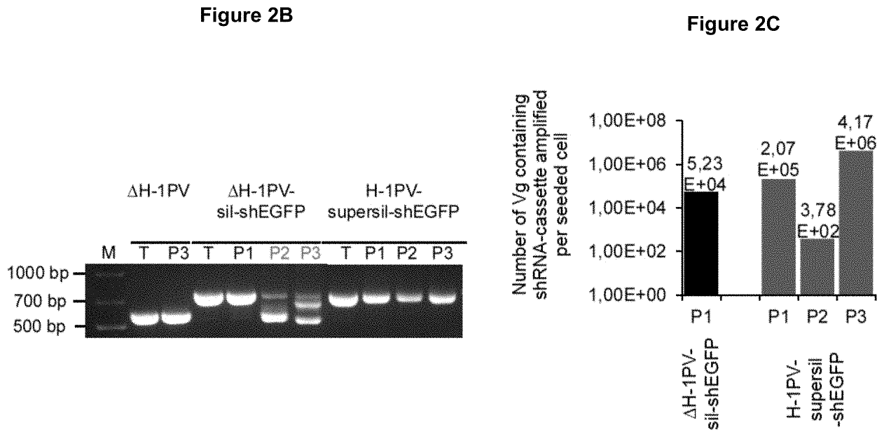

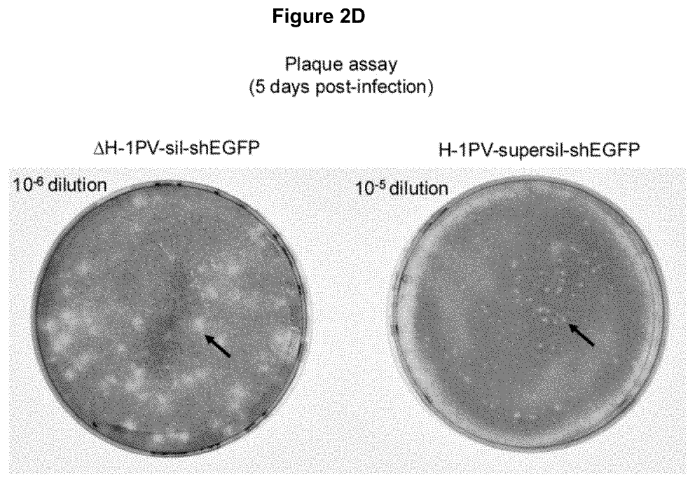

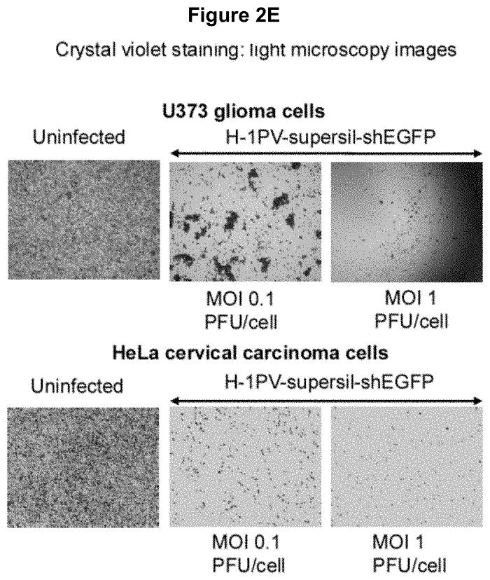

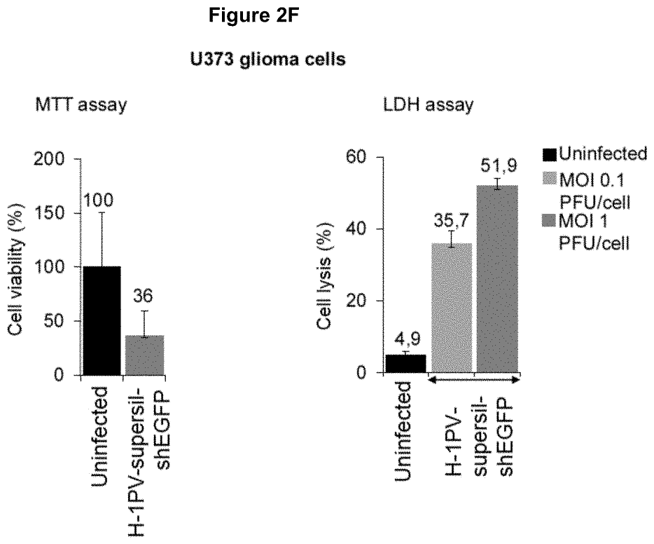

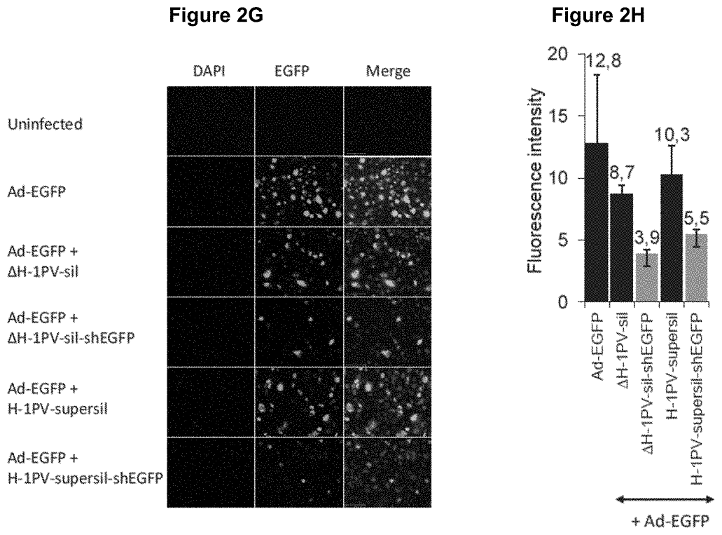

We hypothesized that ΔΔH-1PV-v1 displaying an additional deletion in its genome may have superior cargo capacity in comparison with the ΔH-1PV-sil and keep the shRNA expression cassette in its genome more stably integrated. To verify this hypothesis, the same shRNA expression cassette present in ΔH-1PV-shEGFP including a shRNA sequence against the EGFP gene, was cloned into the ΔΔH-PV-v1 genome within its untranslated region (FIG. 2A), thus generating H-1PV-supersilencer shEGFP (H-1PV-supersil-shEGFP). HEK293T cells were transfected with plasmids carrying the ΔH-1PV-sil-shEGFP or H-1PV-supersil-shEGFP viral genome and then viruses produced via transfection were used as an inoculum for the infection of NB324K cells. Three rounds of amplification (P1, P2 and P3) were performed in NB324K according to the scheme depicted in FIG. 1B cells. At the end of each passage complete lyse of the cells was observed as an indicator of good virus production.

However, for H-1PV-supersil-shEGFP cell lysis was achieved after 5-8 days in comparison with the 3-4 days observed with the ΔH-1PV-sil-shEGFP virus, suggesting a slower kinetic of production. At the end of each passage, virus was isolated from cell lysates and viral DNA analyzed by PCR for the presence of the shRNA expression cassette. We discovered that the cassette remained integrated in the H-1PV-supersil-shEGFP genome for all the three passages while in ΔH-1PV-sil-shEGFP was lost already after the first rounds of amplification during passage 2 (FIG. 2B). We then measured for how many amplifications cycles the shRNA expression cassette was kept into the genome of ΔH-1PV-silshEGFP and H-1PV-supersil-shEGFP genomes. qRT-PCR was used to titer the virus used for the inoculum (INPUT) and that generated at the end of each production cycle (OUTPUT). We found that the cassette was kept into the H-1PV-supersilencer backbone for at least 8 logs longer in comparison with ΔH-1PV-silencer (FIG. 2C). We confirmed by plaque assay that H-1PV-supersil-shEGFP was fully infectious although plaques were smaller than those generated by ΔH-1PV-sil-shEGFP, confirming a slower kinetic of replication (FIG. 2D). We also demonstrated that the insertion of the cassette did not affect the ability of the virus to exert oncolytic activities (FIGS. 2E and F).

We then verified the capacity of H-1PV-supersil-shEGFP to silence gene expression. U373 cells were infected with “first generation” ΔH-1PV-sil (without shRNA) or ΔH-1PV-sil-shEGFP viruses or the corresponding “second generation” H-1PV-supersil or H-1PV-supersil-shEGFP versions. After 20 hours, cells were then superinfected with a recombinant adenovirus expressing EGFP. Cells were grown for further 72 hours, fixed and then analyzed by fluorescent microscopy. EGFP intensity signal was measured using the Fiji-ImageJ software. Decreased EGFP signal intensity was observed in cells infected with ΔH-1PV-sil-shEGFP and H-1PV-supersil-shEGFP viruses (FIGS. 2G and H), showing that the two viruses are efficient in gene silencing.

### Example 3: LuIII-Silencer

Following OVs treatment the insurgence of neutralizing antibodies inactivates the virus, hampering its infectivity and consequently the anti-tumor efficacy. Furthermore, anti-viral antibodies preclude the repetitive systemic administration of OVs. Combination of different viruses to be used in co-sequential way may overcome this limitation and improve clinical outcome. We explored the possibility of using other protoparvoviruses for gene silencing. As an example, LuIII is selected, which has been described to have promising oncolytic activities. LuIII is an autonomous parvovirus of the genus Parvovirus.

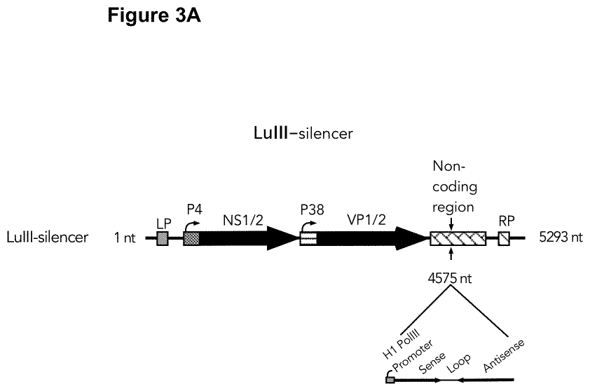

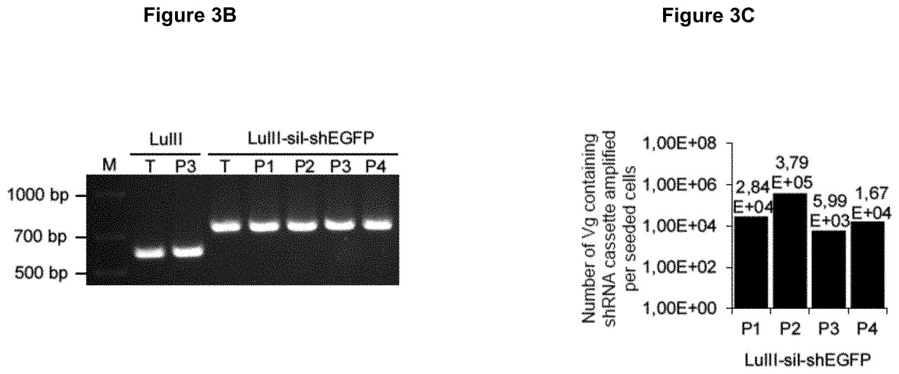

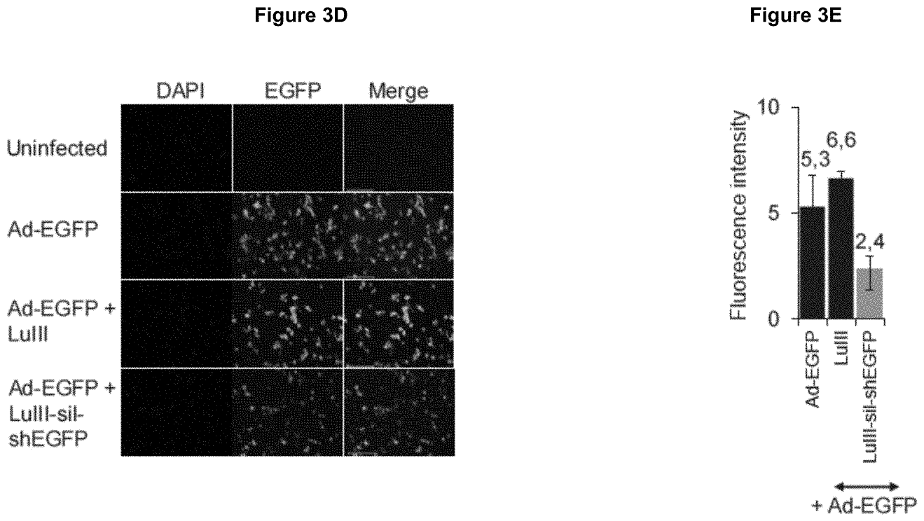

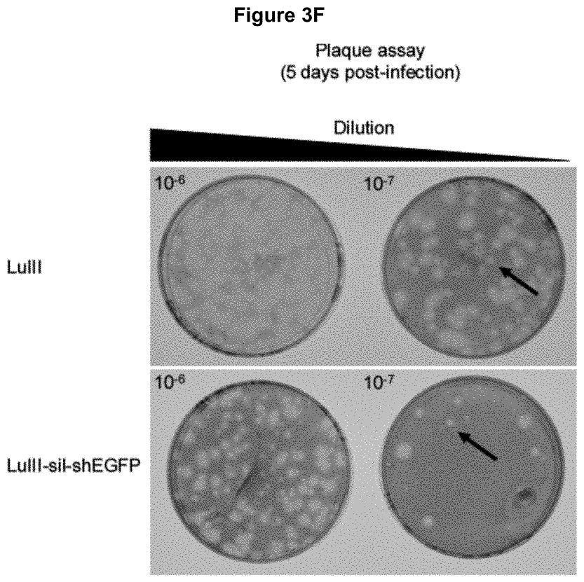

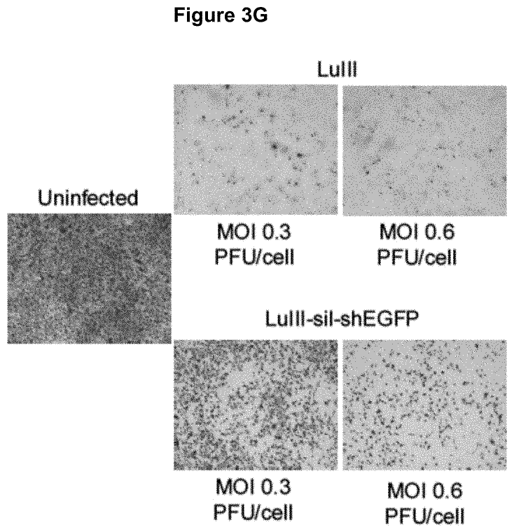

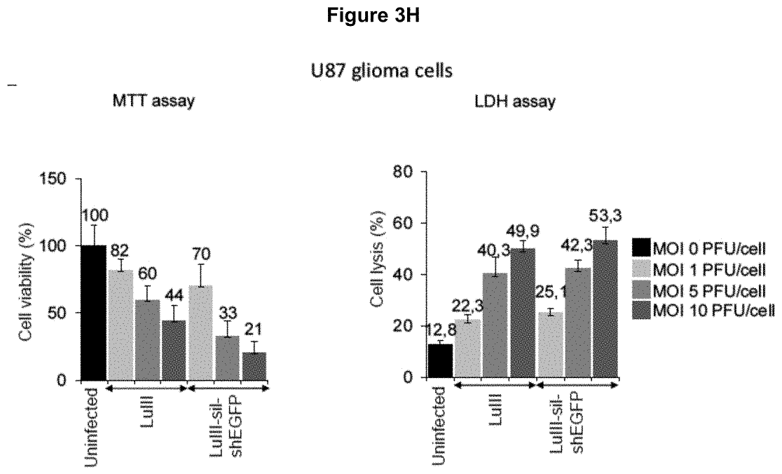

While H-1PV and LuIII belong to the same genus, these viruses differ sufficiently in their capsids so that antibodies developed against H-1PV will not recognize and neutralize LuIII (and viceversa), allowing co-sequential use of these viruses in cancer therapy (in combination or not with other anticancer therapies). However, to date there is no published report describing the engineering of LuIII genome. It is therefore unknown whether and in which position of its genome, LuIII tolerates the insertion of a shRNA expression cassette. A DNA fragment including the 4288-5135 nt of the LuIII genome in which the shRNA expression cassette containing a shRNA targeting the EGFP gene was inserted at position 4575 (untranslated region). The DNA fragment was synthesized and cloned into the pLuIII molecular infection plasmid to replace the analogue LuIII region, thus generating LuIII-sil-shEGFP (FIG. 3A). Similar to H-1PV based clones, the plasmid was used for the transfection of HEK293T cells. The virus particles produced in HEK293T cells were used as inoculum and propagated in NB324K for four passages, following a protocol similar to that described in FIG. 1B. During virus propagation, the shRNA expression cassette, remained stably integrated into the LuIII viral genome (FIGS. 3B and C). The engineered virus through the expression of the shRNAs was able to silence gene expression (FIGS. 3D and E). At the same time, LuIII-sil-shEGFP, similarly to its parental virus, maintained ability to replicate and be fully infectious (formation of plaques) (FIG. 3F), to exert oncolytic activities and disturbances on cell viability (FIGS. 3G and H).

It is noted that the LuIII genome is analogous to H-1PV, and the deletions described herein regarding H-1PV can also be introduced into the LuIII genome to increase its cargo capacity.

### Example 4: Silencing TRAF3IP2 Using H-1PV-Silencer and H-1PV-Supersilencer

As an example of a gene involved in tumor formation, progression and metastasis that could be a potential target of ΔH-1PV-silencer and H-1PV-supersilencer viruses in cancer treatment, TRAF3IP2 gene is selected as the target to test the silencing effect of these engineered viruses.

It has been reported that the adaptor protein NF-κB activator 1 (Act-1), encoded by the TRAF3TP2 gene, plays an essential role in the development and progression of GBM. Inventor's recent results show that shRNA-based knock-down of TRAF3IP2 by means of lentiviruses is a promising strategy against GBM. In particular, it was shown that administration of TRAF3IP2KD shRNA-LV into the right lateral ventricle of NIH-III nude mice carrying a tumor derived from U87 cells in the left somatosensory cortex resulted in marked reduction of tumor size and limited angiogenesis compared to scrambled shRNA-LV treatment.

More recently, it is reported that silencing of TRAF3IP2 is very effective in arresting the development of breast cancer in a xenograft mouse models of human breast cancer. Interestingly, tumor suppression was also associated with a decreased number of micrometastases, which supports a role of TRAF3IP2 in metastasis-formation.

Three shRNA sequences were designed to be inserted into the engineered parvoviruses: one corresponding to the sequence developed in Alt et al. (ASO1), and two de novo synthesized herein. The three shRNAs target different regions of the TRAF3IP2 gene, namely exons 2, 7, 9 and 10 (see Table 1). The three shRNA sequences were cloned into both ΔH-1PV-silencer and H-1PV-supersilencer genomes as described in the Materials and Methods section.

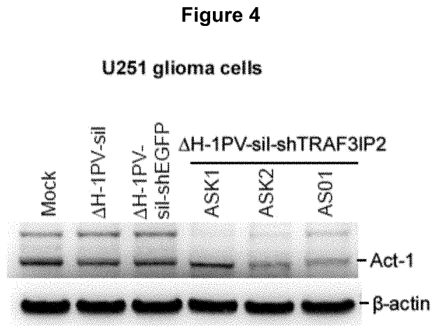

ΔH-1PV-silencer expressing these shRNAs were effective in down-regulating the expression of TRAF3IP2 in U251 glioblastoma cell line as revealed by a strong decrease of Act-1 protein levels (in particular viruses expressing ASK2 and ASO1 shRNAs) (FIG. 4). Similar results were obtained with H-1PVsupersil-shTRAF3IP2 viruses, and further evaluation will confirm the effectiveness of cancer treatment using the oncolytic viruses with TRAF3IP2 silencer.

This disclosure enables synergistic anti-tumor mode of action by providing oncolytic properties and gene silencing through the engineered parvoviruses. The sequential use of viruses with different immunogenic properties in patients can also improve anti-cancer efficacy, as the patient may develop antibodies against one virus over time to render the anti-tumor action less effective. Inventors also envision the possibility to modify the untranslated region of LuIII (e.g. by introducing small deletions) that could further improve its cargo capacity.

### Example 5: Efficient Silencing of TRAF3IP2 and PD-L1 Gene Expression Using LuIII-Silencer

In the previous example, we showed that LuIII-sil-shEGFP maintains a shRNA expression cassette, stably integrated in its genome for multiple replicative rounds (FIG. 3) providing proof-of-concept that LuIII wt has superior packaging capacity than its related H-1PV. The new-engineered virus is efficient in gene silencing (FIG. 3), thus paving the way for the use of LuIII based parvoviruses in gene silencing applications. To further corroborate this discovery, we used LuIII-silencer for the silencing of two cancer related genes, namely TRAF3IP2 and PD-L1. Two shRNA expression cassettes targeting the TRAF3IP2 gene, and one targeting the PD-L1 gene (Table 1), previously inserted into the ΔΔH-1PV genome, were also inserted into the untranslated region of the LuIII genome (FIG. 3A), thus generating LuIII-sil-shTRAF3IP2(ASO1), LuIII-sil-shTRAF3IP2(ASK2), and LuIII-sil-shPD-L1. HEK293T cells were transfected with plasmids carrying the LuIII-sil-shTRAF3JP2(ASO1), LuIII-sil-shTRAF3IP2(ASK2), and LuIII-sil-shPD-L1 recombinant viral genomes or with a plasmid carrying the LuIII wt genome. The virus particles produced via transfection were used as an inoculum for the infection of NB324K cells. Three rounds of virus amplification (P1, P2 and P3) were performed in NB324K cells according to the scheme illustrated in FIG. 1B. At the end of each passage complete lysis was observed after 2-4 days in cells infected with all the three recombinant LuIII viruses similarly to that observed with LuIII wt virus (data not shown). These results provide evidence that the insertion of the cassette did not affect the kinetics of production.

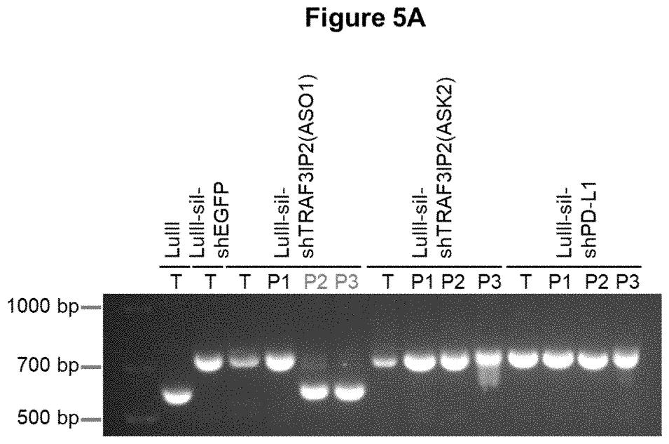

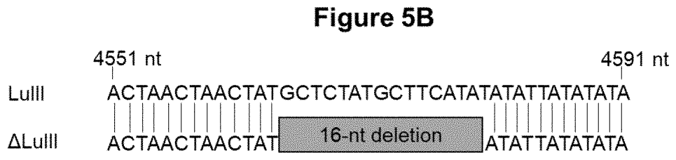

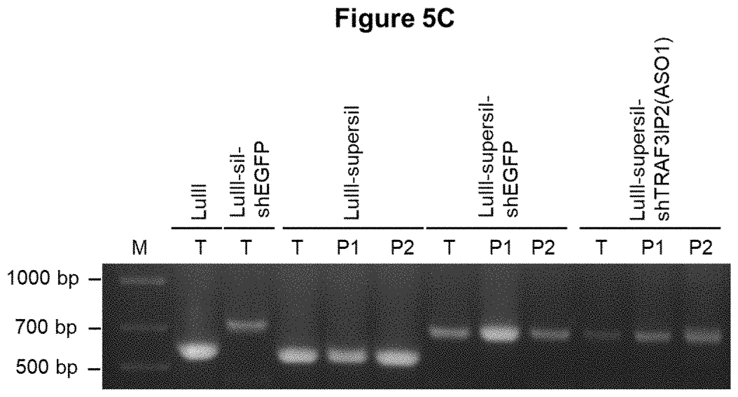

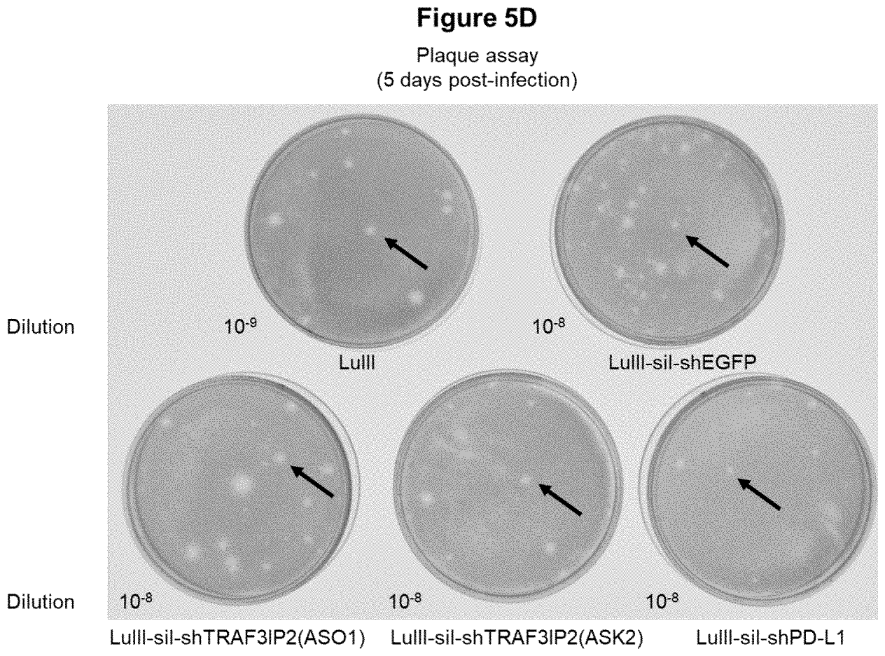

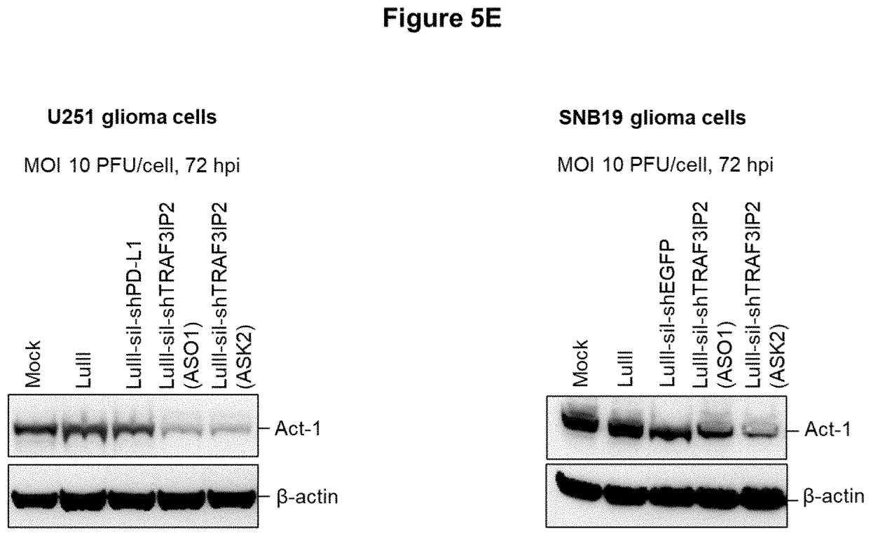

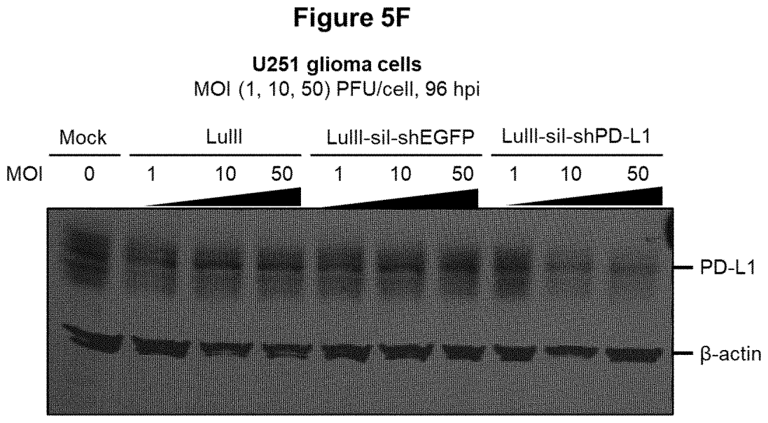

At the end of each passage, virus particles were purified from cell lysates and viral DNA analyzed by RT-PCR for the presence of the shRNA expression cassette. Confirming previous results obtained with LuIII-sil-shEGFP, the cassette remained stably integrated for all the three passages into the LuIII-sil-shTRAF3IP2(ASK2), and LuIII-sil-shPD-L1 viral genomes (FIG. 5A). However, unexpectedly some cassette instability was observed for the LuIII-sil-shTRAF3IP2(ASO1) virus during passage 2 (FIG. 5A). The observed difference in cassette stability of shRNA sequences targeting TRAF3TP2 gene provides clues that also the nucleotide sequence of the shRNA itself (the three viruses differ only for the shRNA sequences) may interfere with the shRNA expression cassette stability, probably affecting the tertiary structure of the viral DNA and thereby its correct packaging. Remarkably, also in the case of LuIII we found that together with the shRNA expression cassette, adjacent to the cassette insertional site, a stretch of sixteen nucleotides (nt) of the viral genome was also lost (position from 4564 to 4579), thus generating a novel LuIII with a shorter genome (ΔLuIII) (FIG. 5B). We anticipate that, following the same approach described in this invention for H-1PV, the new ΔLuIII variant could be also used as a backbone for the insertion of the shRNA expression cassette, probably resulting in a LuIII recombinant virus in which the shRNA expression cassette containing this specific shRNA sequence remains more stably integrated in its genome. Indeed, we show that the shTRAF3IP2(ASO1) expression cassette once inserted in the ΔLuIII (thus generating pLuIII-supersilencer plasmids), is kept for longer time into the viral genome FIG. 5C).

We then confirmed by plaque assay that the LuIII-silencer expressing shRNAs were fully infectious forming plaques of similar size to those generated by LuIII, confirming a similar kinetic of replication (FIG. 5D).

We then verified by Western blotting the capacity of these viruses to silence the expression of target genes. U251 or SNB19 glioma cell lines were infected with LuIII wt, LuIII-sil-shEGFP (negative controls), LuIII-sil-shTRAF3IP2(ASO1), LuIII-sil-shTRAF3IP2(ASK2) or LuIII-sil-shPD-L1. Decreased steady state levels of the Act-1 protein were observed in cells infected with LuIII-sil-shTRAF3IP2(ASO1) and LuIII-sil-shTRAF3IP2(ASK2) viruses but not in cells infected with control viruses (FIGS. 5E and F), demonstrating specific gene silencing. Similarly, decreased steady state levels of the PD-L1 protein were observed in cells infected with LuIII-sil-shPD-L1 virus but not with other control viruses (FIG. 5G) confirming efficient and specific gene expression silencing using LuIII based vectors.

### Example 6: LuIII-Silencer Expressing shRNAs Against TRAF3IP2 Gene have Enhanced Oncolytic Activity while Safe for Normal Cells

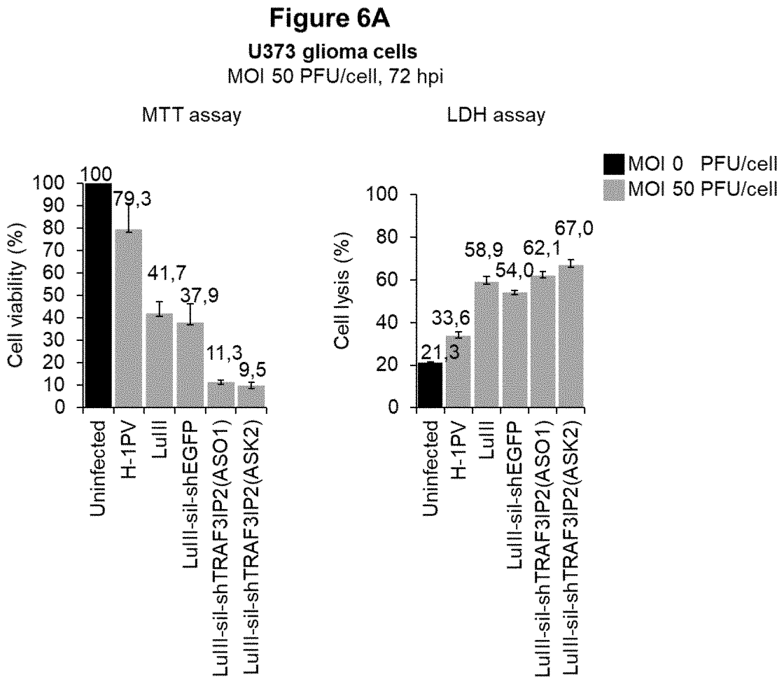

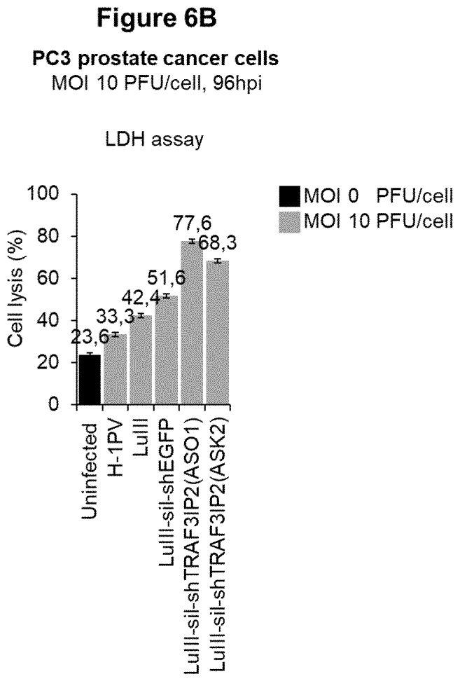

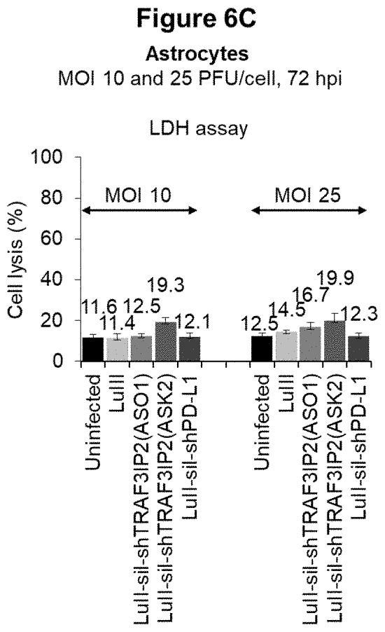

We then investigated whether the insertion of the cassettes targeting the TRAF3IP2 gene would confer the virus an enhanced oncolytic activity. U373 glioma and PC3-prostate cancer derived cell lines were infected with wild type H-1PV (the virus used in clinical trials), wild type LuIII, LuIII-sil-shEGFP (expressing shRNAs targeting EGFP, used here as a negative control), LuIII-sil-shTRAF3IP2(ASO1) and LuIII-sil-shTRAF3TP2(ASK2), both targeting the TRAF3IP2 gene. Cell viability and cell lysis were assessed by MTT and LDH assays respectively. In agreement with previous results, LuIII showed superior antiproliferative activity than H-1PV in glioma derived cell lines (19). Remarkably, this antiproliferative effect was further enhanced in viruses expressing shRNAs against the TRAF3IP2 gene. These results provide evidence that it is possible to increase the anticancer activity of parvoviruses by inserting into their genomes a shRNA expression cassette targeting the TRAF3IP2 gene (FIGS. 6A and B).

It is known that parvoviruses specifically kill cancer cells but not normal non-transformed cells, displaying an excellent safety profile. To verify whether safety is preserved for the parvoviruses expressing shRNAs we used primary astrocytes cell cultures. Astrocytes were infected with wild type LuIII, LuIII-sil-shTRAF3IP2(ASO1) and LuIII-sil-shTRAF3IP2(ASK2), both targeting the TRAF3IP2 gene, LuIII-sil-shPD-L1 targeting PD-L1 gene and after 3 days processed for LDH assay. Only a very marginal increase in the cell lysis was observed in astrocytes infected with LuIII expressing shRNAs against TRAF3IP2 or PD-L1 genes (FIG. 6C), proving that the insertion of the cassette (and the consequent expression of the shRNAs) does not change the safety profile of the viruses substantially.

### Example 7: Neutralizing Antibodies Generated Against H-1PV do not Block the Infection of LuIII and Vice Versa

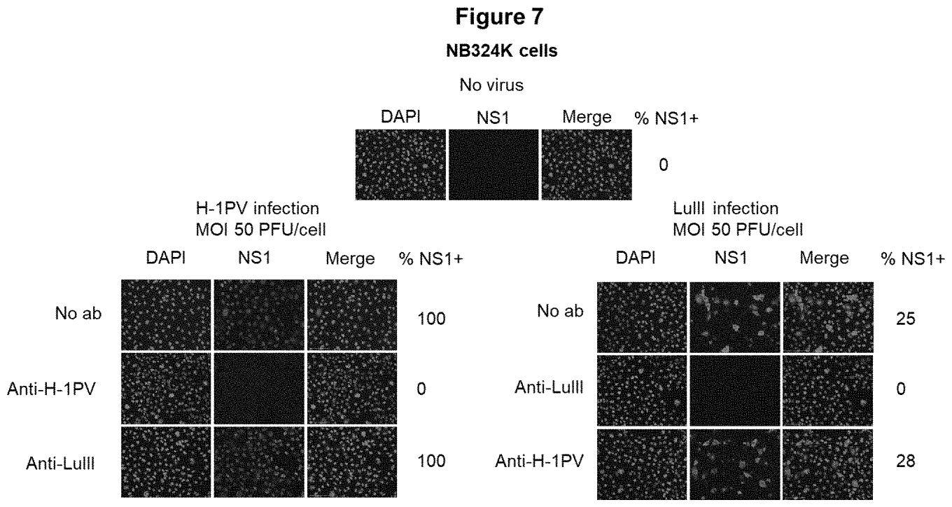

One of the barriers that limits oncolytic virus-immuno therapy is the insurgence of neutralizing anti-viral antibodies observed upon virus treatment, which hampers virus infectivity and consequently anti-tumor efficacy, especially in case of re-treatment with the same virus. One solution to overcome this limitation is to use combination of different viruses in co-sequential manner. However, in case of related viruses belonging to the same family, like H-1PV and LuIII, the compatibility of the two viruses to be used in combination remains to be demonstrated as neutralizing antibody generated against one virus may still be able to at least partially block the infection of the other related virus. In this study, for the first time, we investigated the possibility of using H-1PV and LuIII in co-sequential manner. NB324K cells were pretreated with either neutralizing antibodies against H-1PV or LuIII. We observed that neutralizing antibodies against H-1PV fail to block the infection of LuIII and vice-versa (FIG. 7). These results strongly support the clinical use of H-1PV and LuIII in combination therapy.

The following references are incorporated by reference in their entirety for all purposes.
1. Marchini, A., Daeffler, L., Pozdeev, V. I., Angelova, A. & Rommelaere, J. Immune Conversion of Tumor Microenvironment by Oncolytic Viruses: The Protoparvovirus H-1PV Case Study. Front. Immunol. 10, 1848, doi:10.3389/fimmu.2019.01848 (2019).
2. Bretscher, C. & Marchini, A. H-1 Parvovirus as a Cancer-Killing Agent: Past, Present, and Future. Viruses 11 doi:10.3390/v11060562 (2019).
3. Marchini, A., Bonifati, S., Scott, E. M., Angelova, A. L. & Rommelaere, J. Oncolytic parvoviruses: from basic virology to clinical applications. Virol J 12, 6, doi:10.1186/s12985-014-0223-y (2015).
4. Geletneky, K. et al. Oncolytic H-1 Parvovirus Shows Safety and Signs of Immunogenic Activity in a First Phase I/IIa Glioblastoma Trial. Mol Ther, doi:10.1016/j.ymthe.2017.08.016 (2017).
5. Hartley, A., Kavishwar, G., Salvato, I. & Marchini, A. A Roadmap for the Success of Oncolytic Parvovirus-Based Anticancer Therapies. Annu Rev Virol, doi:10.1146/annurevvirology-012220-023606 (2020).
6. Tam, P. & Astell, C. R. Replication of minute virus of mice minigenomes: novel replication elements required for MVM DNA replication. Virology 193, 812-824, doi:10.1006/viro.1993.1190 (1993).
7. Astell, C. R. et al. Minute virus of mice cis-acting sequences required for genome replication and the role of the trans-acting viral protein, NS-1. Prog Nucleic Acid Res Mol Biol 55, 245-285, doi:10.1016/s0079-6603(08)60196-8 (1996).
8. Illarionova, A., Rommelaere, J., Leuchs, B. & Marchini, A. Modified parvovirus useful for gene silencing. EP2620503 and EP2807259; Australia AU2013211755, Japan 5901796. (2015).
9. Weiss, N., Stroh-Dege, A., Rommelaere, J., Dinsart, C. & Salome, N. An in-frame deletion in the NS protein-coding sequence of parvovirus H-1PV efficiently stimulates export and infectivity of progeny virions. J Virol 86, 7554-7564, doi:10.1128/JVI.00212-12 (2012).
10. De Oliveira A. P. et al. H-1PV expressing shRNAs targeting CDK9 have enhanced anticancer activity. Patent application (2016).

11. Agbandje-McKenna M & Chapman M S., p. Correlating structure with function in viral capsid. In Kerr J R, Cotmore S F, Bloom M E, Linden R M, Parrish C R (ed), Parvoviruses. Hodder Arnold, London, United Kingdom., 125-139 (2006).
12. Halder, S. et al. Structural characterization of H-1 parvovirus: comparison of infectious virions to empty capsids. J Virol 87, 5128-5140, doi:10.1128/JVI.03416-12 (2013).
13. Tattersall, P. & Bratton, J. Reciprocal productive and restrictive virus-cell interactions of immunosuppressive and prototype strains of minute virus of mice. J Virol. 46(3), 944-955, doi:doi:10.1128/JVI.46.3.944-955.1983 (1983).
14. Alt, E. U. et al. TRAF3IP2, a novel therapeutic target in glioblastoma multiforme. Oncotarget 9, 29772-29788, doi:10.18632/oncotarget.25710 (2018).
15. Cotmore, S. F. & Tattersall, P. Genome packaging sense is controlled by the efficiency of the nick site in the right-end replication origin of parvoviruses minute virus of mice and LuIII. J Virol 79, 2287-2300, doi:10.1128/JVI.79.4.2287-2300.2005 (2005).
16. El-Andaloussi, N. et al. Generation of an adenovirus-parvovirus chimera with enhanced oncolytic potential. J Virol 86, 10418-10431, doi:10.1128/JVI.00848-12 (2012).
17. Li, J. et al. Synergistic combination of valproic acid and oncolytic parvovirus H-1PV as a potential therapy against cervical and pancreatic carcinomas. EMBO Mol Med 5, 1537-1555, doi:10.1002/emmm.201302796 (2013).
18. Rasband, W. S. ImageJ. U. S. National Institutes of Health, Bethesda, Md., USA, http://imagej.nih.gov/ij/(1997-2011.).
19. Paglino, J. C., Ozduman, K. & van den Pol, A. N. LuIII parvovirus selectively and efficiently targets, replicates in, and kills human glioma cells. J Virol 86, 7280-7291, doi:10.1128/JVI.00227-12 (2012).
20. Gaffen, S. L., Jain, R., Garg, A. V. & Cua, D. J. The IL-23-IL-17 immune axis: from mechanisms to therapeutic testing. Nat Rev Immunol 14, 585-600, doi:10.1038/nri3707 (2014).
21. Israel, A. The IKK complex, a central regulator of NF-kappaB activation. Cold Spring Harb Perspect Biol 2, a000158, doi:10.1101/cshperspect.a000158 (2010).
22. Thiefes, A. et al. Simultaneous blockade of NFkappaB, INK, and p38 MAPK by a kinaseinactive mutant of the protein kinase TAK1 sensitizes cells to apoptosis and affects a distinct spectrum of tumor necrosis factor [corrected] target genes. J Biol Chem 280, 27728-27741, doi:10.1074/jbc.M411657200 (2005).
23. Alt, E. U. et al. Targeting TRAF3IP2, Compared to Rab27, is More Effective in Suppressing the Development and Metastasis of Breast Cancer. Nature Sci Rep 10, 8834, doi:10.1038/s41598-020-64781-z (2020).
24. Geletneky K, Hajda J, Angelova A L, Leuchs B, Capper D, Bartsch A J, Neumann J O, Schoning T, Husing J, Beelte B, Kiprianova I, Roscher M, Bhat R, von Deimling A, Bruck W, Just A, Frehtman V, Lobhard S, Terletskaia-Ladwig E, Fry J et al. (2017) Oncolytic H-1 Parvovirus Shows Safety and Signs of Immunogenic Activity in a First Phase I/IIa Glioblastoma Trial. Mol Ther
25. Kestler J, Neeb B, Struyf S, Van Damme J, Cotmore S F, D'Abramo A, Tattersall P, Rommelaere J, Dinsart C, Cornelis J J (1999) cis requirements for the efficient production of recombinant DNA vectors based on autonomous parvoviruses. Hum Gene Ther 10: 1619-32.
26. Weiss N, Stroh-Dege A, Rommelaere J, Dinsart C, Salome N (2012) An in-frame deletion in the NS protein-coding sequence of parvovirus H-1PV efficiently stimulates export and infectivity of progeny virions. J Virol 86: 7554-64
27. Brockhaus K et al. (1996), Nonstructural proteins NS2 of minute virus of mice associate in vivo with 14-3-3 protein family members. Journal of virology vol. 70,11: 7527-34.
28. Kulkarni A et al (2021), Oncolytic H-1 parvovirus binds to sialic acid on laminins for cell attachment and entry. Nat Commun. June 22; 12(1):3834.
29. Schindelin, J., Arganda-Carreras, I., Frise, E., Kaynig, V., Longair, M., Pietzsch, T., Cardona, A. (2012). Fiji: an open-source platform for biological-image analysis. Nature Methods, 9(7), 676-682.

What is claimed is:

1. An engineered parvovirus, the parvovirus having a left palindromic terminal sequence, a coding sequence, a non-coding region, and a right palindromic terminal sequence, wherein:
a) the coding sequence comprising a first sequence encoding non-structural proteins and a second sequence encoding structural capsid proteins; and
b) a first deletion is located between the first sequence and the second sequence and a second deletion is located within the untranslated region;

2. The engineered parvovirus of claim 1 in which the parvovirus is a rodent protoparvovirus.

3. The engineered parvovirus of claim 2, wherein the parvovirus is H-1PV or LuIII.

4. The engineered parvovirus of claim 3, further comprising a silencer expressing sequence inserted in the non-coding region.

5. The engineered parvovirus of claim 4, wherein the engineered parvovirus is H-1PV and comprises a first deletion at nucleotide 2022 to 2135 of the wildtype H-1PV genome and a second deletion at position 4659-4693 of the wild type H-1PV genome.

6. The engineered parvovirus of claim 5, wherein the silencer expression sequence is a silencer expression cassette inserted at nucleotide 4659 of the wild type H-1PV genome.

7. The engineered parvovirus of claim 6 wherein the silencer expressing sequence is a shRNA expressing cassette.

8. The engineered parvovirus of claim 3, wherein the shRNA expressing cassette comprising a sense sequence of a target sequence, a loop, and an antisense sequence of the target sequence and a promoter regulated by a RNA polymerase II or a RNA polymerase III.

9. The engineered parvovirus of claim 8, wherein the shRNA expressing cassette comprises a RNA-polymerase III H1 promoter

10. The engineered parvovirus of claim 8, wherein the shRNA silences the expression of a target gene related to cancer, wherein the target gene is oncogenes, anti-apoptotic genes, a gene critical for tumor cell growth, metastasis, acquisition of drug resistance, angiogenesis or aberrant expression of an immunomodulatory gene or a gene encoding an immunomodulatory checkpoint, cytokine, growth factor, enzyme or transcription factor.

11. The engineered parvovirus of claim 8, wherein the target sequence is a portion of TRAF3IP2 gene or PD-L1 gene.

12. A composition for treating a cancer, comprising effective amount of the engineered virus of claim 4, and a pharmaceutically acceptable carrier.

13. The composition of claim 12, wherein composition is formulated for intravascular injection, parenteral administration, intratumoral administration or intranasal administration.

14. An engineered LuIII parvovirus having a left palindromic terminal sequence, a coding sequence, a non-coding region, and a right palindromic terminal sequence, wherein a deletion is located within the non-coding region.

15. The engineered LuIII parvovirus of claim 14, wherein the deletion includes nucleotides 4564 to 4579 of a wild type LuIII virus.

16. The engineered LuIII parvovirus of claim 14, further comprising a silencer expression sequence inserted in the non-coding region and remains stably integrated during extensive virus propagation.

17. The engineered LuIII parvovirus of claim 16, wherein the silencer expression sequence is inserted at position 4575 of the wild type LuIII genome, or position 4563 of the non-coding region of the engineered LuIII parvovirus.

18. The engineered LuIII parvovirus of claim 17, wherein the silencer expression sequence is a shRNA expression cassette comprising a sense sequence of a portion of a target gene, a loop, an antisense sequence of the portion of the target gene, and a promoter or promoter region regulated by a RNA polymerase II or a RNA polymerase III.

19. The engineered LuIII parvovirus of claim 18, wherein the shRNA expressing cassette contains a RNA-polymerase III H1 promoter.

20. The engineered LuIII parvovirus of claim 16, wherein the shRNA sequence silences the expression of a target gene related to cancer, wherein the target gene is oncogenes, anti-apoptotic genes, a gene critical for tumor cell growth, metastasis, acquisition of drug resistance, angiogenesis or aberrant expression of an immunomodulatory gene or a gene encoding an immunomodulatory checkpoint, cytokine, growth factor, enzyme or transcription factor.

21. The engineered LuIII parvovirus of claim 18, wherein the target gene is TRAF3IP2 or PD-L1.

22. A composition for treating a cancer, comprising an effective amount of the engineered LuIII parvovirus of claim 21, and a pharmaceutically acceptable carrier.

23. The composition of claim 22, wherein the composition is formulated for intravascular injection, parenteral administration, intratumoral administration or intranasal administration.

24. A method of treating a cancer, comprising administering to a subject an effective amount of the engineered parvoviruses of claim 4, and a pharmaceutically acceptable carrier.

25. The method of claim 24 wherein the cancer is glioblastoma.

26. The method of claim 24, wherein the engineered parvovirus is an engineered H-1PV, the engineered H-1PV comprising a first deletion at nucleotide 2022 to 2135 of the wildtype H-1PV genome and a second deletion at position 4659-4693 of the wild type H-1PV genome.

27. A method of treating a cancer, comprising administering to a subject an effective amount of the engineered LuIII parvovirus of claim 16, and a pharmaceutically acceptable carrier.

28. The method of claim 27, wherein the engineered parvovirus is administered in combination with other anticancer agents.

29. The method of claim 27, further comprising administering to the subject a second oncolytic virus or a viral vector.

30. The method of 29, wherein the second oncolytic virus is an engineered H-1PV parvovirus, the parvovirus having a left palindromic terminal sequence, a coding sequence, a non-coding region, and a right palindromic terminal sequence, wherein: the coding sequence comprising a first sequence encoding non-structural proteins and a second sequence encoding structural capsid proteins; and a first deletion is located between the first sequence and the second sequence and a second deletion is located within the untranslated region, wherein the engineered H-1PV parvovirus further comprising a silencer expressing sequence inserted in the non-coding region.

31. The method of claim 30, wherein the engineered parvovirus and the engineered LuIII parvovirus and the engineered H-1PV parvovirus are administered co-sequentially or simultaneously.

32. The method of claim 30, wherein the engineered LuIII parvovirus and the engineered H-1PV parvovirus silence the same target gene.


SEQUENCE LISTING (23 sequences)

SEQ ID NO: 1 - DNA, 19 bp, Artificial Sequence
  Sense sequence of portion of ASK1
tagcaatcagccccaaata

SEQ ID NO: 2 - DNA, 19 bp, Artificial Sequence
  Antisense sequence of portion of ASK1
tatttggggctgattgcta

SEQ ID NO: 3 - DNA, 18 bp, Artificial Sequence
  Sense sequence of portion of ASK2
accatacccaagtcgttg

SEQ ID NO: 4 - DNA, 19 bp, Artificial Sequence
  Antisense sequence of portion of ASK2
caactgacttgggtatggt

SEQ ID NO: 5 - DNA, 20 bp, Artificial Sequence
  Sense sequence of portion of ASO1
agaaggagcatgtgcccacc

SEQ ID NO: 6 - DNA, 20 bp, Artificial Sequence
  Antisense sequence of portion of ASO1
ggtgggcacatgctccttct

SEQ ID NO: 7 - DNA, 25 bp, Artificial Sequence
  Sense sequence of portion of PD-L1
ggacaagcagtgaccatcaagtcct

SEQ ID NO: 8 - DNA, 25 bp, Artificial Sequence
  Antisense sequence of portion of PD-L1
aggacttgatggtcactgcttgtcc

SEQ ID NO: 9 - DNA, 19 bp, Artificial Sequence
  Sense sequence of portion of EGFP
gctggagtacaactacaac

SEQ ID NO: 10 - DNA, 19 bp, Artificial Sequence
  Antisense sequence of portion of EGFP
gttgtagttgtactccagc

SEQ ID NO: 11 - DNA, 22 bp, Artificial Sequence
  Forward PCR primer for LuIII
aatgctccaggtcagcttctgg

SEQ ID NO: 12 - DNA, 23 bp, Artificial Sequence
  Reverse PCR primer for LuIII
gtccctaacattcagtctaaggg

SEQ ID NO: 13 - DNA, 1077 bp, Artificial Sequence
  H-1PV-supersil-shEGFP [targeting the EGFP gene]
taacatgcattatactaatgtttttaacagctatggtccacttagtgcatttcctcatccagatcccatttatccaaatggacaaatttgggacaaagaattggacctggaacacaaacctagactacacgtaactgcaccatttgtttgtaaaaacaacccaccaggtcaactatttgttcacttggggcctaatctgactgaccaatttgacccaaacagcacaactgtttctcgcattgttacatatagcactttttactggaagggtattttgaaattcaaagccaaactaagaccaaatctgacctggaatcctgtataccaagcaaccacagactctgttgccaattcttacatgaatgttaagaaatggctcccatctgcaactggcaacatgcactctgatccattgatttgtagacctgtgcctcacatgacatactaaccaaccaactatgtttctctgtttgcttcacataatacttaaactaactagactacaacataaaaatatacacttaataataggaattcatatttgcatgtcgctatgtgttctgggaaatcaccataaacgtgaaatgtctttggatttgggaatcttataagttctgtatgagaccactcggatccgctggagtacaactacaacttcaagagagttgtagttgtactccagcttttttggaagcggccgctaaaaaataatagaacttttggaataaatatagttagttggttaatgttagatagaatataaaaagattttgtattttaaaataaatatagttagttggttaatgttagatagaatataaaaagattttgtatttgggaaataaaaagggtggttgggtggttggttggtactcccttagactgaatgttagggaccaaaaaaataataaaataattaaaatgaacaaggactactgtctattcagttgaccaactgaacctatagtatcactatgtttttagggtgggggggtgggagatacatacgttcgctatggaccaagtggtaccggttggttgctaagctcgaacaagacggctaagccggtccggttg

SEQ ID NO: 14 - DNA, 1053 bp, Artificial Sequence
  H-1PV-supersil-shTRAF3IP2(ASK1) [targeting the TRAF3IP2 gene]
taacatgcattatactaatgtttttaacagctatggtccacttagtgcatttcctcatccagatcccatttatccaaatggacaaatttgggacaaagaattggacctggaacacaaacctagactacacgtaactgcaccatttgtttgtaaaaacaacccaccaggtcaactatttgttcacttggggcctaatctgactgaccaatttgacccaaacagcacaactgtttctcgcattgttacatatagcactttttactggaagggtattttgaaattcaaagccaaactaagaccaaatctgacctggaatcctgtataccaagcaaccacagactctgttgccaattcttacatgaatgttaagaaatggctcccatctgcaactggcaacatgcactctgatccattgatttgtagacctgtgcctcacatgacatactaaccaaccaactatgtttctctgtttgcttcacataatacttaaactaactagactacaacataaaaatatacacttaataatagatatttgcatgtcgctatgtgttctgggaaatcaccataaacgtgaaatgtctttggatttgggaatcttataagttctgtatgagaccacccggtagcaatcagccccaaatactcgagtatttggggctgattgctattttttgtaaaaaataatagaacttttggaataaatatagttagttggttaatgttagatagaatataaaaagattttgtattttaaaataaatatagttagttggttaatgttagatagaatataaaaagattttgtatttgggaaataaaaagggtggttgggtggttggttggtactcccttagactgaatgttagggaccaaaaaaataataaaataattaaaatgaacaaggactactgtctattcagttgaccaactgaacctatagtatcactatgtttttagggtgggggggtgggagatacatacgttcgctatggaccaagtggtaccggttggttgctaagctcgaacaagacggctaagccggtccggttg

SEQ ID NO: 15 - DNA, 1053 bp, Artificial Sequence
  H-1PV-supersil-shTRAF3IP2(ASK2) [targeting the TRAF3IP2 gene]
taacatgcattatactaatgtttttaacagctatggtccacttagtgcatttcctcatccagatcccatttatccaaatggacaaatttgggacaaagaattggacctggaacacaaacctagactacacgtaactgcaccatttgtttgtaaaaacaacccaccaggtcaactatttgttcacttggggcctaatctgactgaccaatttgacccaaacagcacaactgtttctcgcattgttacatatagcactttttactggaagggtattttgaaattcaaagccaaactaagaccaaatctgacctggaatcctgtataccaagcaaccacagactctgttgccaattcttacatgaatgttaagaaatggctcccatctgcaactggcaacatgcactctgatccattgatttgtagacctgtgcctcacatgacatactaaccaaccaactatgtttctctgtttgcttcacataatacttaaactaactagactacaacataaaaatatacacttaataatagatatttgcatgtcgctatgtgttctgggaaatcaccataaacgtgaaatgtctttggatttgggaatcttataagttctgtatgagaccacccggaccatacccaagtcagttgctcgagcaactgacttgggtatggtttttttgtaaaaaataatagaacttttggaataaatatagttagttggttaatgttagatagaatataaaaagattttgtattttaaaataaatatagttagttggttaatgttagatagaatataaaaagattttgtatttgggaaataaaaagggtggttgggtggttggttggtactcccttagactgaatgttagggaccaaaaaaataataaaataattaaaatgaacaaggactactgtctattcagttgaccaactgaacctatagtatcactatgtttttagggtgggggggtgggagatacatacgttcgctatggaccaagtggtaccggttggttgctaagctcgaacaagacggctaagccggtccggttg

SEQ ID NO: 16 - DNA, 1055 bp, Artificial Sequence
  H-1PV-supersil-shTRAF3IP2(ASO1) [targeting the TRAF3IP2 gene]
taacatgcattatactaatgtttttaacagctatggtccacttagtgcatttcctcatccagatcccatttatccaaatggacaaatttgggacaaagaattggacctggaacacaaacctagactacacgtaactgcaccatttgtttgtaaaaacaacccaccaggtcaactatttgttcacttggggcctaatctgactgaccaatttgacccaaacagcacaactgtttctcgcattgttacatatagcactttttactggaagggtattttgaaattcaaagccaaactaagaccaaatctgacctggaatcctgtataccaagcaaccacagactctgttgccaattcttacatgaatgttaagaaatggctcccatctgcaactggcaacatgcactctgatccattgatttgtagacctgtgcctcacatgacatactaaccaaccaactatgtttctctgtttgcttcacataatacttaaactaactagactacaacataaaaatatacacttaataatagatatttgcatgtcgctatgtgttctgggaaatcaccataaacgtgaaatgtctttggatttgggaatcttataagttctgtatgagaccacccggagaaggagcatgtgcccaccctcgagggtgggcacatgctccttctttttttgtaaaaaataatagaacttttggaataaatatagttagttggttaatgttagatagaatataaaaagattttgtattttaaaataaatatagttagttggttaatgttagatagaatataaaaagattttgtatttgggaaataaaaagggtggttgggtggttggttggtactcccttagactgaatgttagggaccaaaaaaataataaaataattaaaatgaacaaggactactgtctattcagttgaccaactgaacctatagtatcactatgtttttagggtgggggggtgggagatacatacgttcgctatggaccaagtggtaccggttggttgctaagctcgaacaagacggctaagccggtccggttg

SEQ ID NO: 17 - DNA, 1017 bp, Artificial Sequence
  LuIII-sil-shEGFP [targeting the EGFP gene]
taggctagcacctaacttgactgaccagtatgatcctaatagttctaacctatctagaattgtcacctatggcaccttcttctggaagggcaaactaactctaaaagcaaagatgagacctaatgctacttggaacccagtcttccaaataagtgctaccaaccaaggaaccaatgactacatgagcattgaaagatggttaccaactgctactggcaacataacaaatgtgcctctgctttctagacctgttgctagaaacacttactaactaactatgctctatgcttatatttgcatgtcgctatgtgttctgggaaatcaccataaacgtgaaatgtctttggatttgggaatcttataagttctgtatgagaccacgctggagtacaactacaacttcaagagagttgtagttgtactccagcttttttggaacatatatattatatatattattatactaactaaccatgtttactcttacattacttcatataatattaagactaataaaaatacaacatagaaatataatattacatatagatataaagaatagaataatatggtacttacttactgttagaaataatagaacttttggaataacaagatagttagttggtttatgttatatagaatataagaagatgatgtacaaagaataaaagggtgggagggtggttggttggtactcccttagactgaatgttagggaccaaaaaaataataaaattcttgaaaacccaacaaggactactgtcatattcagtgaaccaactgaaccattagtatcaatatgattttagggtgggggggtgggagatacatatgttcactatggaccaactggtactggttggttgctctgctccaaccaaccagaccggctctgccggtctggttggttgagcgcaaccaaccagtaccagttggtccatagtgaacatatgtatctcccacccccccaccctaaaaacatattgatactaatgggatccccgggtaccgagc

SEQ ID NO: 18 - DNA, 770 bp, Artificial Sequence
  LuIII-sil-shTRAF3IP2(ASO1) [targeting the TRAF3IP2 gene]
taggctagcacctaacttgactgaccagtatgatcctaatagttctaacctatctagaattgtcacctatggcaccttcttctggaagggcaaactaactctaaaagcaaagatgagacctaatgctacttggaacccagtcttccaaataagtgctaccaaccaaggaaccaatgactacatgagcattgaaagatggttaccaactgctactggcaacataacaaatgtgcctctgctttctagacctgttgctagaaacacttactaactaactatgctctatgcttatatttgcatgtcgctatgtgttctgggaaatcaccataaacgtgaaatgtctttggatttgggaatcttataagttctgtatgagaccacccggagaaggagcatgtgcccaccctcgagggtgggcacatgctccttctttttttgcatatatattatatatattattatactaactaaccatgtttactcttacattacttcatataatattaagactaataaaaatacaacatagaaatataatattacatatagatataaagaatagaataatatggtacttacttactgttagaaataatagaacttttggaataacaagatagttagttggtttatgttatatagaatataagaagatgatgtacaaagaataaaagggtgggagggtggttggttggtactcccttagactgaatgttagggaccaaaaaaataataaaattcttgaaaacccaacaaggactactgtcata

SEQ ID NO: 19 - DNA, 768 bp, Artificial Sequence
  LuIII-sil-shTRAF3IP2(ASK2) [targeting the TRAF3IP2 gene]
taggctagcacctaacttgactgaccagtatgatcctaatagttctaacctatctagaattgtcacctatggcaccttcttctggaagggcaaactaactctaaaagcaaagatgagacctaatgctacttggaacccagtcttccaaataagtgctaccaaccaaggaaccaatgactacatgagcattgaaagatggttaccaactgctactggcaacataacaaatgtgcctctgctttctagacctgttgctagaaacacttactaactaactatgctctatgcttatatttgcatgtcgctatgtgttctgggaaatcaccataaacgtgaaatgtctttggatttgggaatcttataagttctgtatgagaccacccggaccatacccaagtcagttgctcgagcaactgacttgggtatggtttttttgcatatatattatatatattattatactaactaaccatgtttactcttacattacttcatataatattaagactaataaaaatacaacatagaaatataatattacatatagatataaagaatagaataatatggtacttacttactgttagaaataatagaacttttggaataacaagatagttagttggtttatgttatatagaatataagaagatgatgtacaaagaataaaagggtgggagggtggttggttggtactcccttagactgaatgttagggaccaaaaaaataataaaattcttgaaaacccaacaaggactactgtcata

SEQ ID NO: 20 - DNA, 782 bp, Artificial Sequence
  LuIII-sil-shPD-L1 [targeting the PD-L1 gene]
taggctagcacctaacttgactgaccagtatgatcctaatagttctaacctatctagaattgtcacctatggcaccttcttctggaagggcaaactaactctaaaagcaaagatgagacctaatgctacttggaacccagtcttccaaataagtgctaccaaccaaggaaccaatgactacatgagcattgaaagatggttaccaactgctactggcaacataacaaatgtgcctctgctttctagacctgttgctagaaacacttactaactaactatgctctatgcttatatttgcatgtcgctatgtgttctgggaaatcaccataaacgtgaaatgtctttggatttgggaatcttataagttctgtatgagaccacccggggacaagcagtgaccatcaagtcctctcgagaggacttgatggtcactgcttgtccttttttggacatatatattatatatattattatactaactaaccatgtttactcttacattacttcatataatattaagactaataaaaatacaacatagaaatataatattacatatagatataaagaatagaataatatggtacttacttactgttagaaataatagaacttttggaataacaagatagttagttggtttatgttatatagaatataagaagatgatgtacaaagaataaaagggtgggagggtggttggttggtactcccttagactgaatgttagggaccaaaaaaataataaaattcttgaaaacccaacaaggactactgtcata

SEQ ID NO: 21 - DNA, 606 bp, Artificial Sequence
  delta-LuIII (16 nucleotide deletion in LuIII genome at 4564-4579 position)
taggctagcacctaacttgactgaccagtatgatcctaatagttctaacctatctagaattgtcacctatggcaccttcttctggaagggcaaactaactctaaaagcaaagatgagacctaatgctacttggaacccagtcttccaaataagtgctaccaaccaaggaaccaatgactacatgagcattgaaagatggttaccaactgctactggcaacataacaaatgtgcctctgctttctagacctgttgctagaaacacttactaactaactatatattatatatattattatactaactaaccatgtttactcttacattacttcatataatattaagactaataaaaatacaacatagaaatataatattacatatagatataaagaatagaataatatggtacttacttactgttagaaataatagaacttttggaataacaagatagttagttggtttatgttatatagaatataagaagatgatgtacaaagaataaaagggtgggagggtggttggttggtactcccttagactgaatgttagggaccaaaaaaataataaaattcttgaaaacccaacaaggactactgtcata

SEQ ID NO: 22 - DNA, 754 bp, Artificial Sequence
  LuIII-supersil-shEGFP [targeting the EGFP gene]
taggctagcacctaacttgactgaccagtatgatcctaatagttctaacctatctagaattgtcacctatggcaccttcttctggaagggcaaactaactctaaaagcaaagatgagacctaatgctacttggaacccagtcttccaaataagtgctaccaaccaaggaaccaatgactacatgagcattgaaagatggttaccaactgctactggcaacataacaaatgtgcctctgctttctagacctgttgctagaaacacttactaactaactatatatttgcatgtcgctatgtgttctgggaaatcaccataaacgtgaaatgtctttggatttgggaatcttataagttctgtatgagaccacgctggagtacaactacaacttcaagagagttgtagttgtactccagcttttttggaaatattatatatattattatactaactaaccatgtttactcttacattacttcatataatattaagactaataaaaatacaacatagaaatataatattacatatagatataaagaatagaataatatggtacttacttactgttagaaataatagaacttttggaataacaagatagttagttggtttatgttatatagaatataagaagatgatgtacaaagaataaaagggtgggagggtggttggttggtactcccttagactgaatgttagggaccaaaaaaataataaaattcttgaaaacccaacaaggactactgtcata

SEQ ID NO: 23 - DNA, 754 bp, Artificial Sequence
  LuIII-supersil-shTRAF3IP2(ASO1) [targeting the TRAF3IP2 gene]
taggctagcacctaacttgactgaccagtatgatcctaatagttctaacctatctagaattgtcacctatggcaccttcttctggaagggcaaactaactctaaaagcaaagatgagacctaatgctacttggaacccagtcttccaaataagtgctaccaaccaaggaaccaatgactacatgagcattgaaagatggttaccaactgctactggcaacataacaaatgtgcctctgctttctagacctgttgctagaaacacttactaactaactatatatttgcatgtcgctatgtgttctgggaaatcaccataaacgtgaaatgtctttggatttgggaatcttataagttctgtatgagaccacccggagaaggagcatgtgcccaccctcgagggtgggcacatgctccttctttttttgatattatatatattattatactaactaaccatgtttactcttacattacttcatataatattaagactaataaaaatacaacatagaaatataatattacatatagatataaagaatagaataatatggtacttacttactgttagaaataatagaacttttggaataacaagatagttagttggtttatgttatatagaatataagaagatgatgtacaaagaataaaagggtgggagggtggttggttggtactcccttagactgaatgttagggaccaaaaaaataataaaattcttgaaaacccaacaaggactactgtcata